(19) United States
(12) Patent Application Publication
(10) Pub. No.: US 2014/0058282 A1
(43) Pub. Date: Feb. 27, 2014

(54) SYSTEM AND METHOD FOR GASTRO-INTESTINAL ELECTRICAL ACTIVITY

(71) Applicants: Gregory B. O'Grady, Auckland (NZ); Leo K. Cheng, Auckland (NZ); Andrew J. Pullan, Auckland (NZ); Peng Du, Auckland (NZ); Nira Paskaranandavadivel, Auckland (NZ); Timothy R. Angeli, Holland, MI (US); Gianrico Farrugia, Rochester, MN (US); Samuel J. Asirvatham, Rochester, MN (US)

(72) Inventors: Gregory B. O'Grady, Auckland (NZ); Leo K. Cheng, Auckland (NZ); Andrew J. Pullan, Auckland (NZ); Peng Du, Auckland (NZ); Nira Paskaranandavadivel, Auckland (NZ); Timothy R. Angeli, Holland, MI (US); Gianrico Farrugia, Rochester, MN (US); Samuel J. Asirvatham, Rochester, MN (US)

(73) Assignees: Mayo Foundation for Medical Education and Research, Rochester, MN (US); Auckland Uniservices Limited, Auckland (NZ)

(21) Appl. No.: 13/880,041

(22) PCT Filed: Oct. 18, 2011
(86) PCT No.: PCT/NZ11/00217

§ 371 Date: Sep. 6, 2013

Related U.S. Application Data
(60) Provisional application No. 61/394,171, filed on Oct. 18, 2010.

(51) Int. Cl. A61B 5/00; A61B 5/0488

(52) U.S. Cl. CPC A61B 5/42; A61B 5/0488
USPC 600/546

(57) ABSTRACT

A gastro-electrical activity mapping system and method may comprise a catheter insertable through a natural orifice into the gastro-intestinal (GI) tract and comprising an array of electrodes for contacting an interior surface of a section of the GI tract to detect electrical potentials at multiple electrodes, and comprises a signal analysis and mapping system arranged to receive and process electrical signals from multiple electrodes of the array and spatially map GI smooth muscle electrical activity as an activation time map, a velocity map, or an amplitude map, which may be in the form of contour plots and may be mapped on an anatomical computer model of at least the section of the GI tract and may be animated.

### FIELD OF INVENTION

The invention relates to a system and method for analysis of gastro-intestinal electrical activity.

### BACKGROUND

Gastroparesis is a condition in which the stomach typically fails to empty properly after a meal, leading to symptoms of early satiety, bloating, pain, nausea, vomiting, and in severe cases, malnutrition. Functional dyspepsia is a condition characterised by symptoms of ‘chronic indigestion’, lasting at least weeks to months, and which may include bloating, nausea and pain after eating. Delayed gastric emptying occurs in 25-40% of functional dyspepsia. Gastro-oesophageal reflux disease (GORD) is a condition involving the reflux of acidic gastric contents into the oesophagus accompanied by symptoms, primarily heartburn.

Gastric motility is controlled by an underlying bioelectrical activity, termed slow waves, and dysrhythmias of this electrical activity contribute to gastric dysfunction. Studies using electrogastrography (cutaneous gastric electrical measurements of uncertain reliability) and/or few ‘sparse’ electrodes have suggested that dysrhythmias occur routinely in gastroparesis, commonly in functional dyspepsia, and also in certain sub-populations of patients with GORD (eg, those who also have dyspepsia and those who experience regurgitation symptoms (8)). Gastric dysrhythmias may also occur in other functional disorders including cyclical vomiting syndrome, and morning sickness of pregnancy. However, the nature, mechanisms and clinical significance of gastric dysrhythmias has remained poorly understood, due to the limitations of the technologies previously used to assess them.

Peristaltic activity in the GI tract is coordinated by a propagating electrical activity termed slow waves. GI slow waves are initiated and spread via networks of interstitial cells of Cajal (ICCs), which are coupled to the smooth muscle layers in the GI tract wall. In the human stomach, slow waves originate at a pacemaker site high on the greater curvature, and propagate toward the antrum at a normal frequency of approximately three cycles per minute(cpm). Three cpm is the ‘intrinsic’ frequency of cells only in the pacemaker region. More distal areas of the stomach have been shown to intrinsically operate at lower frequencies (1.5-2 cpm) when isolated from the pacemaker region. In an intact network, therefore, all cells are synchronised to the fastest frequency in the syncytium in a process called ‘entrainment’.

The stomach may come to operate at abnormally high frequencies (termed ‘tachygastria’) or sometimes abnormally low frequencies (‘bradygastria’) and different regions of the stomach can become ‘uncoupled’, causing dynamically-competing wavefronts that collide and/or abnormal patterns of activity. Among most important of these abnormalities is tachygastria, because it has been recognised most often in disease states. There are two recognised types of tachygastria: irregular and regular. The standard conception of tachygastria is that a specific ‘focus’ of cells come to operate at a faster frequency than the rest of the stomach. The mechanisms behind this standard theory are poorly understood, but one rationale is that prostaglandins (locally acting physiological messenger hormones) might serve to raise the intrinsic frequency of a patch of slow waves above their normal level.

### SUMMARY OF INVENTION

In broad terms in one aspect the invention comprises a system for analysis of gastrointestinal-electrical activity comprising:

a catheter insertable into the gastro-intestinal (GI) tract, or other electrode-carrier for contacting an exterior serosal surface of the GI tract, and comprising one or more electrodes for contacting a surface of a section of the GI tract to detect electrical potentials at multiple electrodes,

a processing system arranged to receive and process electrical signals from the one or more electrodes of the array and spatially map the GI smooth muscle electrical activity at said section of the GI tract and identify as indicative of disease including (but not limited to) gastroparesis and/or functional dyspepsia or as useful in the diagnosis of disease mechanisms in gastro-oesophageal reflux disease and other gastro-intestinal motility disorders or nausea and vomiting disorders any one of or any of in combination:
propagation of re-entrant or non re-entrant GI electrical activity upwardly within the GI tract such as from the pylorus or antrum or other lower part of the corpus of the stomach in a direction towards an upper part of the corpus, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, for example as a result of a conduction problem such as a partial or complete conduction block (the abnormal cessation of propagating slow wave wavefront), and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, for example as a result of a focal event or events or ectopic activity/activities (i.e. slow waves arising from a site other than the normal pacemaker), or as a result of an escape activity (an ectopic activity arising after a delay to the normal excitation), and other than at the normal pacemaker site in the stomach, and
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

In some embodiments the system is arranged to display any one or more of an activation time map indicative of the propagation of electrical activity, a propagating wavefront animation, a velocity map indicative of slow wave velocity and/or direction, an amplitude map of slow wave signal amplitudes across the stomach, and a dysrhythmia map of the GI electrical activity.

In some embodiments the system may comprise a reference database indicative of geometries of one or more sections of the GI tract and related characteristics such as subject height and sex relating to each geometry, and the system is arranged to select a best-fit geometry from the database for each subject under study and optionally modify the selected geometry.

In broad terms in a further aspect the invention comprises a method for mapping GI electrical activity which comprises acquiring electrical potentials from at least one electrode contacting a surface of a section of the GI tract and spatially mapping from the electrical signals GI electrical activity at said section of the GI tract and identifying as indicative of disease including (but not limited to) gastroparesis and/or functional dyspepsia or as useful in the diagnosis of disease mechanisms in gastro-oesophageal reflux disease and other gastro-intestinal motility disorders or nausea and vomiting disorders any one of or any of in combination:
propagation of re-entrant or non re-entrant GI electrical activity upwardly within the GI tract such as from the pylorus or antrum or other lower part of the corpus of the stomach in a direction towards an upper part of the corpus, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, for example as a result of a conduction problem such as a partial or complete conduction block (the abnormal cessation of propagating slow wave wavefront), and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, for example as a result of a focal event or events or ectopic activity/activities (i.e. slow waves arising from a site other than the normal pacemaker), or as a result of an escape activity (an ectopic activity arising after a delay to the normal excitation), and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

In a preferred form said processing of the electrical potential signals detected at the electrodes includes animating the individual propagating waves over a generic or subject-specific anatomical model.

The processing may also include making time activation maps of waves, calculating velocity and amplitude fields from the activation maps, and displaying the activation maps and velocity fields over the anatomical model.

The processing may also include comparing the GI electrical activity to a stored reference database to provide an indication of normal or abnormal GI electrical activity.

In broad terms in another aspect the invention comprises a system arranged to receive and process electrical signals (obtained for example by electrocardiography) relating to GI smooth muscle electrical activity in the GI tract and to identify from said electrical signals as indicative of disease including (but not limited to) gastroparesis and/or functional dyspepsia or as useful in the diagnosis of disease mechanisms in gastro-oesophageal reflux disease and other gastro-intestinal motility disorders or nausea and vomiting disorders any one of or any of in combination:
propagation of re-entrant or non re-entrant GI electrical activity upwardly within the GI tract such as from the pylorus or antrum or other lower part of the corpus of the stomach in a direction towards an upper part of the corpus, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, for example as a result of a conduction problem such as a partial or complete conduction block (the abnormal cessation of propagating slow wave wavefront), and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, for example as a result of a focal event or events or ectopic activity/activities (i.e. slow waves arising from a site other than the normal pacemaker), or as a result of an escape activity (an ectopic activity arising after a delay to the normal excitation), and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

The invention also includes a method which comprises receiving and processing electrical signals (obtained for example by electrogastrography) relating to GI smooth muscle electrical activity in the GI tract to identify from said electrical signals as indicative of disease including (but not limited to) gastroparesis and/or functional dyspepsia or as useful in the diagnosis of disease mechanisms in gastro-oesophageal reflux disease and other gastro-intestinal motility disorders or nausea and vomiting disorders any one of or any of in combination:
propagation of re-entrant or non re-entrant GI electrical activity upwardly within the GI tract such as from the pylorus or antrum or other lower part of the corpus of the stomach in a direction towards an upper part of the corpus, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, for example as a result of a conduction problem such as a partial or complete conduction block (the abnormal cessation of propagating slow wave wavefront), and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, for example as a result of a focal event or events or ectopic activity/activities (i.e. slow waves arising from a site other than the normal pacemaker), or as a result of an escape activity (an ectopic activity arising after a delay to the normal excitation), and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

In electrogastrography (EGG) for gastric dysrhythmias electrodes are placed on the skin to record the distant organ electrical activity.

Normally, slow waves propagate in successive wavefronts that travel longitudinally down the stomach. Circumferential slow wave propagation (slow waves travelling transversely across the stomach) does not normally occur, except for a short distance at the normal pacemaker region, because ring wavefronts are quickly established after slow waves originate at the pacemaker region, such that excitable tissue only remains in the longitudinal organ axis.

The system and method of the invention are intended to be useful particularly in the diagnosis of gastric dysrhythmias including in gastroparesis and functional dyspepsia, and nausea and vomiting disorders, and may also be useful in the diagnosis of disease mechanisms in gastro-oesophageal reflux disease and other gastro-intestinal motility disorders such as small intestinal, colonic and rectal dysmotility disorders, or in other smooth-muscle-lined viscera, including the bladder.

The system of the invention may be employed as an adjunct to upper or lower GI endoscopy.

The system and method of the invention may be useful to guide therapies for gastric dysmotility disorders, including gastric electrical stimulation, targeted ablation of aberrant conduction pathways and targeted drug delivery.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

### BRIEF DESCRIPTION OF THE FIGURES

Embodiments of the invention are further described with reference to the accompanying figures, without intending to be limiting, in which:

FIG. 1 shows a human stomach and illustrates GI smooth muscle electrical or slow wave activity starting at a normal pacemaker area of the stomach in the greater curvature,

FIG. 2 schematically shows a cross-section of the stomach at a time A when that part of the stomach muscle is inactive and a subsequent time B when the same part of the stomach is activated simultaneously as a ring as the wavefront moves down the stomach,

FIG. 3 schematically shows activation of the gastric cross-section during pacing or from the pacemaker site, and that the wavefront passes in opposite directions around the gastric circumference, to an area of quiescence,

FIG. 4 schematically shows upstream and downstream the GI electrical activity forming concentric rings that propagate away from the pacemaker site,

FIG. 5a schematically shows tachygastria originating from a stable ectopic focus of cells operating above their intrinsic frequencies and FIG. 5a schematically shows tachygastria caused by a re-entrant wavefront operating around the anatomical circumference of the stomach,

FIG. 6 shows one embodiment of a gastro-intestinal (GI) mapping catheter, unexpanded,

FIG. 7 shows the GI mapping catheter of FIG. 6, expanded,

FIG. 8 schematically shows intubation of the GI mapping catheter of FIGS. 6 and 7, into the gastric antrum,

FIG. 9 shows the GI mapping catheter of FIGS. 6 and 7 after intubation and expansion until the electrode array of the mapping catheter contacts the mucosal surface of the gastric antrum,

FIG. 9a schematically shows a flexible electrode pad inserted through a keyhole incision made in the abdominal wall and positioned against the external serosal surface of the GI tract,

FIG. 10 shows an example of a user-display on a VDU presented by an EGG system of the invention,

FIG. 11 shows another example of a user-display including actuation time and velocity maps of GI electrical activity, presented by a GI mapping system of the invention,

FIGS. 12a and 12b show further including actuation time and velocity, maps of GI electrical activity, on a stomach model,

FIG. 13 is a flow chart illustrating signal analysis, mapping, and model fitting stages of a preferred embodiment GI mapping system and method of the invention,

FIG. 14 is a flow chart of a preferred embodiment method for GI slow wave activation time identification,

FIG. 15 is a flow chart of a preferred embodiment clustering method for clustering or partitioning of activation times into separate gastric slow wave groups,

FIG. 16a is a pixelated isochronal activation time map or a part thereof and FIG. 16b shows such a smooth filled contour activation time map with isochronal lines,

FIG. 17 shows an isochronal activation time map and a velocity map,

FIG. 18 is a flow chart of a preferred velocity calculation method,

FIG. 19 illustrates identification of a peak and two troughs of a single event in a GEA trace,

FIG. 20 is a flow chart of a preferred amplitude calculation method,

FIG. 21 is a flow chart of an embodiment of a spatial classification scheme for slow wave abnormalities in gastroparesis, and

FIGS. 22 to 37 are referred to in the subsequent examples.

### DETAILED DESCRIPTION OF EMBODIMENTS

In the method and system of the invention GI smooth muscle electrical activity is mapped and any one of or any of the following in combination is identified as indicative of disease including (but not limited to) gastroparesis and/or functional dyspepsia, or nausea and vomiting disorders, or as useful in the diagnosis of disease mechanisms in gastro-oesophageal reflux disease and other gastro-intestinal motility disorders:
propagation of re-entrant or non re-entrant GI electrical activity upwardly within the GI tract such as from the pylorus or antrum or other lower part of the corpus of the stomach in a direction towards an upper part of the corpus, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, for example as a result of a conduction problem such as a partial or complete conduction block (the abnormal cessation of propagating slow wave wavefront), and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, for example as a result of a focal event or events or ectopic activity/activities (i.e. slow waves arising from a site other than the normal pacemaker), or as a result of an escape activity (an ectopic activity arising after a delay to the normal excitation), and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

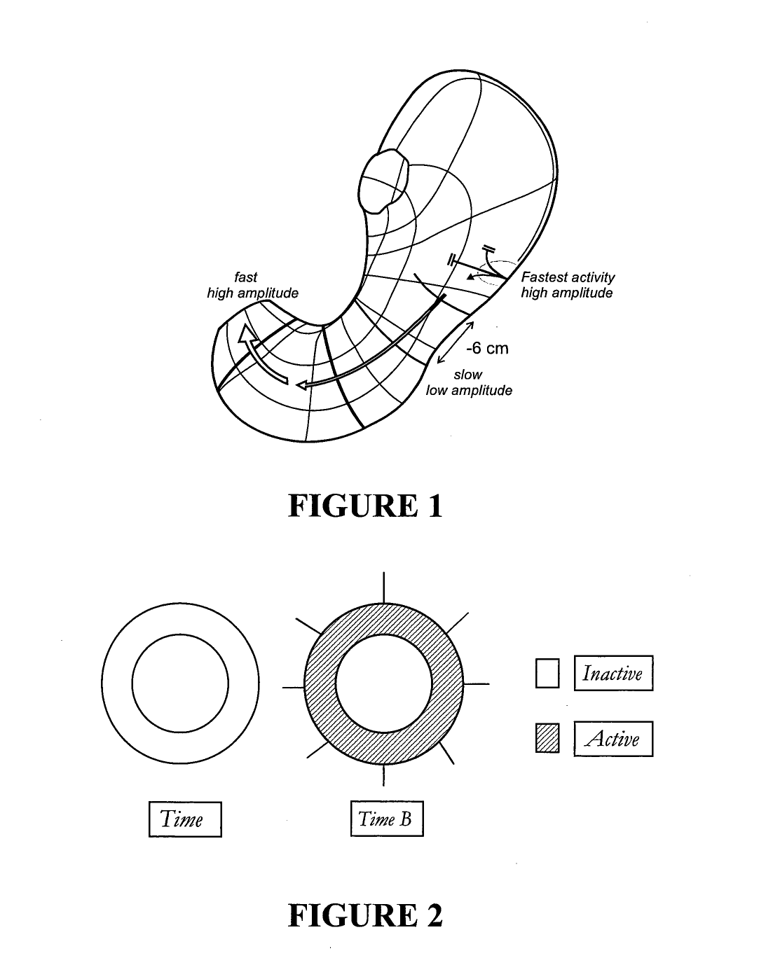

GI smooth muscle electrical activity or slow wave activity starts at a normal pacemaker area of the stomach indicated in FIG. 1 on the greater curvature of the corpus or stomach body. There is initially a small region of circumferential propagation. Activity fans out in all directions, but travels only a very limited distance medially and proximally. Distally, the wavefront spreads out and becomes a ‘ring’ of activation that travels longitudinally down towards the pylorus.

FIG. 2 schematically shows a cross-section of the stomach at a time A when that part of the stomach muscle is inactive and a subsequent time B when the same part of the stomach is activated simultaneously as a ring as the wavefront moves down the stomach. There is no appreciable circumferential propagation of the wavefront around the stomach; activity progresses only longitudinally. In other words, the longitudinal propagation profile allows a nearly zero excitable tissue volume in the circumferential axis, so the circumferential component of the velocity vector is near to zero. Normally, the slow wave propagation velocity is therefore that of the longitudinal propagation velocity (˜3 mm/s in body and proximal antrum, 6 mm/s in distal antrum). However, the tissue propensity is actually anisotropic, because if an excitable tissue volume does exist in the circumferential axis, then circumferential propagation will be more rapid than longitudinal propagation.

Circumferential propagation emerges during a range of gastric dysrhythmias, because if the normal ring wavefronts may be broken by conduction defects, or if aberrant initiation of a wavefront occurs within a field of resting tissue, then excitation is once more free to proceed in the circumferential direction (transversely across the stomach). An increase in extracellular amplitudes accompanies the increase in slow wave velocity because of direct proportionality between velocity and transmembrane current entering the extracellular space. The detection of amplitude and velocity changes therefore now constitutes a novel useful biomarker for detecting, localizing, characterizing and monitoring gastric dysrhythmias. The velocities detected are in the range of 1.5 to 3.5 times higher than normal in the corpus (average 2.5 times) and 1.5 to 3.5 times higher than normal in the antrum (average 2.5 times). The amplitude increase is in the range of 1.5 to 3.5 times higher than normal in the corpus (average 2.5 times and 1.5 to 3.5 times higher than normal in the antrum (average 2.5 times).

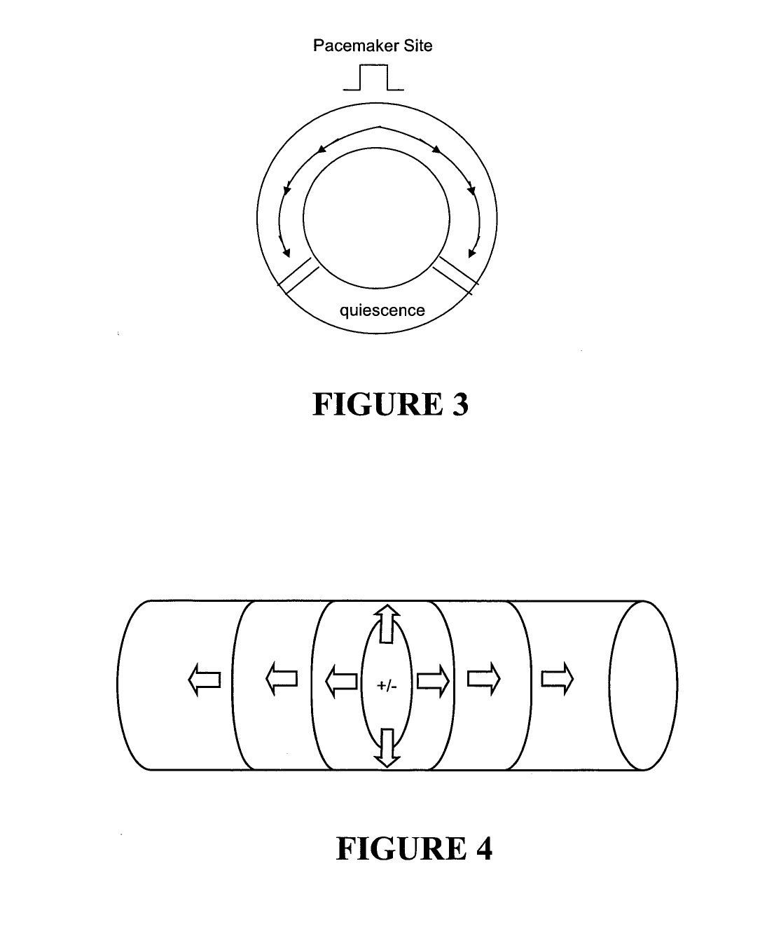

FIG. 3 schematically shows activation of the gastric cross-section during pacing or from the pacemaker site. The wavefront passes in opposite directions around the gastric circumference, away from the pacing site, to an area of quiescence, as shown. This cycle repeats with each stimulus. Upstream and downstream, the activity forms concentric rings that propagate away from the pacing site, as shown in FIG. 4.

During tachygastrias slow waves typically propagate retrograde from the antrum toward the body of the stomach, reversing their normal course. This in turn may lead to reverse contractions, which may be partly responsible for symptom generation. Tachygastrias have also been correlated with dysfunctional gastric smooth muscle contractility.

We have identified a new mechanism for tachygastria based on circumferential re-entry loops.

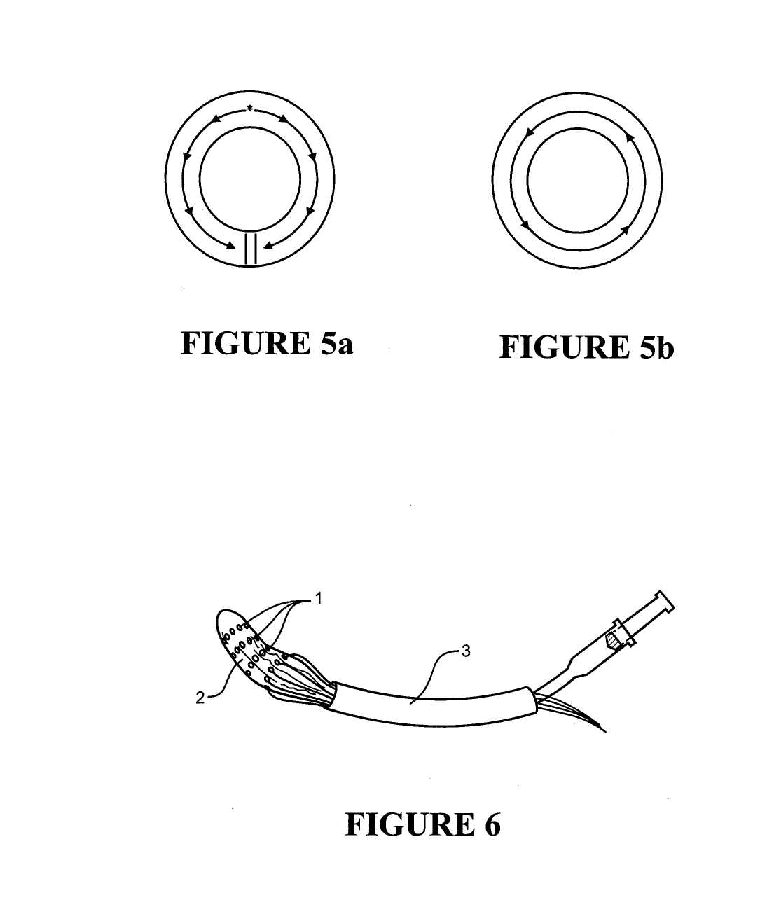

Instead of the tachygastria originating from a stable ectopic focus of cells operating above their intrinsic frequencies as schematically illustrated in FIG. 5a, the tachygastria is caused by a circumferential re-entrant wavefront operating around the anatomical circumference of the stomach as schematically illustrated in FIG. 5b. We believe that this mechanism generates wavefronts that arise at frequency (f) according to the equation:

$$f = \frac{{v}_{c}}{ϕ} ,$$

where νₑ is circumferential velocity and φ is the circumference. These wavefronts will propagate proximally and distally (in a slight ‘cork-screw’ formation) from the point of re-entry according to the equation:

$$d = \frac{{v}_{i}}{{v}_{c}} ϕ ,$$

where νₗ is the longitudinal velocity.

The stability (or instability) of this circumferential re-entrant pattern may be governed by several factors:
It will only arise when there is an excitable tissue volume in the circumferential axis. Circumferential re-entry is then promoted by the fixed path of rapid conduction around the lesser and greater curvatures.
Stable re-entry is possible when the period is longer than the wavelength, else the re-entry wavefront will collide with the refractory tail of the previous cycle and terminate. Stable re-entry is possible when resultant frequency f is >3 cpm (the natural pacemaker frequency). If f<3 cpm, the re-entry will be out-competed by the normal pacemaker, and entrainment by intrinsic activity will follow. In our early experience, ‘tachygastric’ resultant frequencies (arbitrarily defined as ˜>3.7 cpm) only occur in the antrum, where φ is smaller.

Functional re-entrant circuits (operating on the anterior serosal surface of the antrum) have previously been shown to occur in the stomach, but these are a different mechanism and were not shown to be stable. A functional re-entrant wavefront initially propagates in both longitudinal and circumferential directions but ultimately propagates in a loop because of non-uniformity within the tissue, whereas the circumferential re-entry loops wave fronts only propagate in the circumferential direction. Moreover, circumferential re-entry loops have high amplitude and high velocity band comparing to normal detected electrical activities therefore allowing it to be readily observable. The velocities detected are in the range of 2.5 to 3.5 times higher than normal in the corpus and 1.25 to 2.5 times higher than normal in the antrum. The amplitude increase is in the range of 2 to 3.5 times higher than normal in the corpus and 1.25 to 2.5 times higher than normal in the antrum. The circumferential re-entry has greater potential to be a mechanistically stable cause of gastric dysfunction, primarily because of the rapid circumferential conduction pathway.

Re-entry may not be exclusively low in the stomach. For example, it may occur in the corpus as a result of exit-block from the normal pacemaker site, which for example may occur due to degradation of interstitial cell of Cajal networks in diabetes. Re-entry refers to one wave front repeatedly activating a tissue circuit in continuity. An abnormal wavefront may travel in a loop in the circumferential organ axis, along a continuous intrinsic rapid conduction pathway around the lesser and greater curvatures, and then continuously re-enter into that same circumferential tissue circuit.

### System and Method for Identifying Abnormal GI Electrical Activity

A system for mapping gastrointestinal-electrical activity and identifying re-entrant GT electrical loops may comprise a mapping catheter and a processing system to receive and process electrical signals from multiple electrodes, spatially map the GI smooth muscle electrical activity at said section of the GI tract and identify slow wave activity indicative of abnormal GI electrical activity.

### GI Mapping Catheter

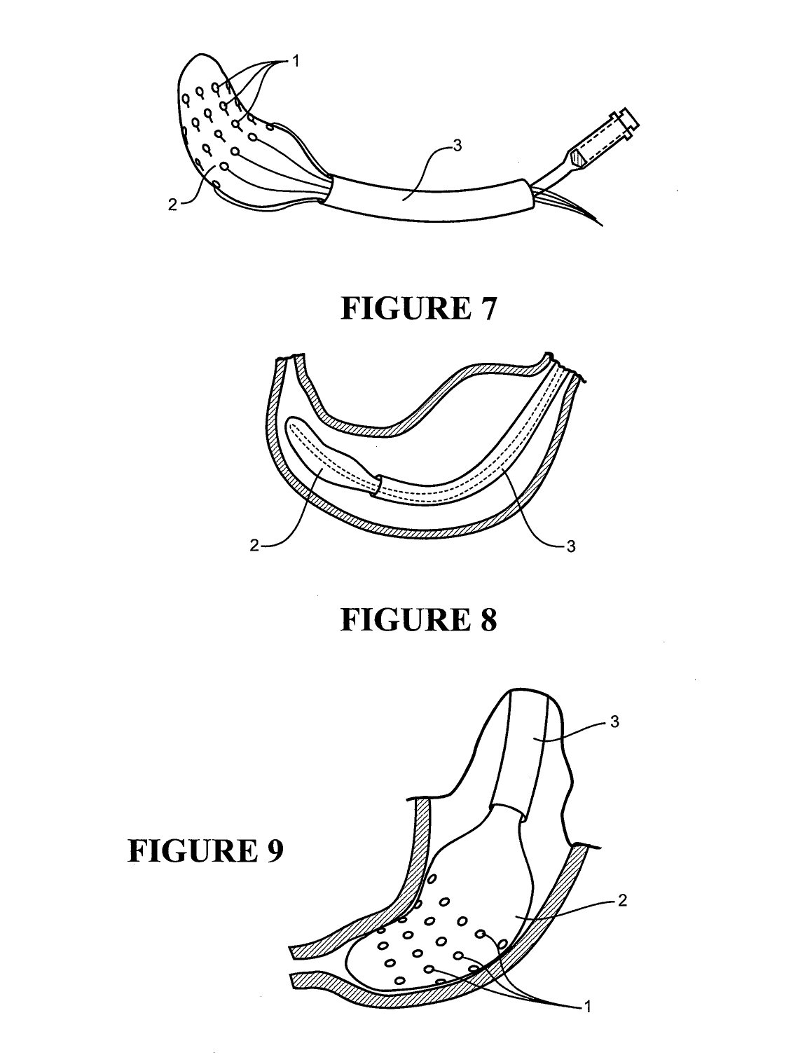

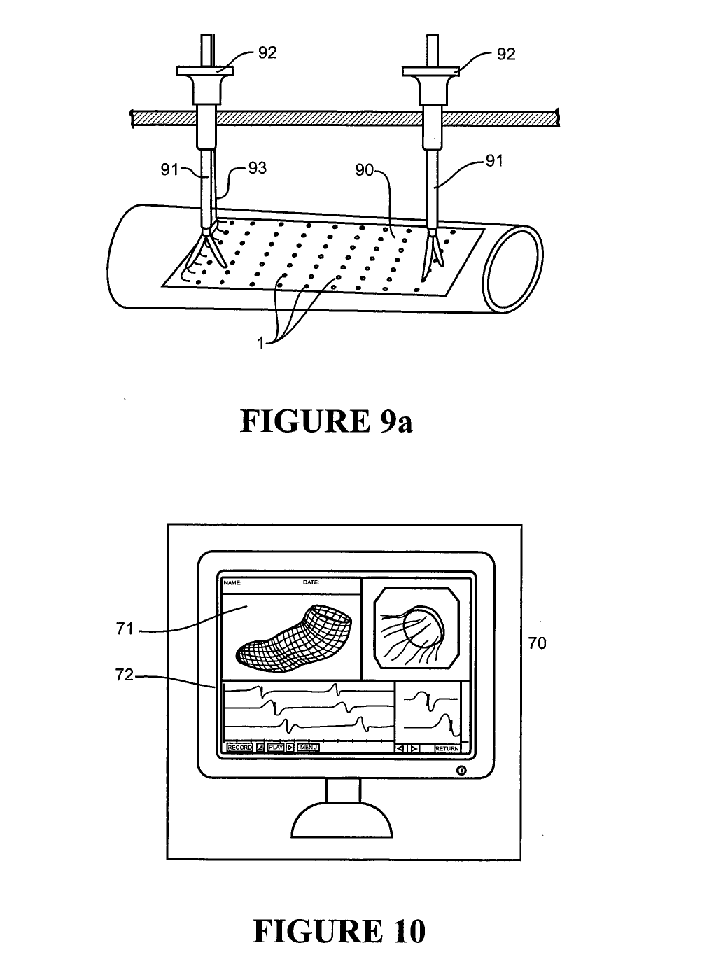

FIGS. 6 and 7 show one form of a mapping catheter useful for mapping GI electrical activity. The catheter comprises one or more and preferably an array of multiple electrodes some indicated at 1 spaced around an expandable electrode carrier comprising an inflatable balloon 2, attached to a nasogastric or oral gastric or similar tube 3. Signal wires or conductors (electrically insulated) one from each electrode 1 pass through the tube 3 from the catheter to exit the proximal end of the nasogastric tube, for example at a plug for coupling the signal lines to electronic instrumentation. FIG. 6 shows the balloon electrode carrier 2 deflated and FIG. 2 shows it inflated. In use the catheter with the balloon 2 deflated is intubated temporarily via a natural orifice, such as via the mouth, into the GI tract and when in position at the desired location, such as in the gastric antrum, gastric corpus, upper small bowel, rectum, large bowel, or bladder, is expanded by inflation through the lumen of the tube 3 until the electrodes 1 or at least some electrodes contact the mucosal surface that part of the GI tract. The catheter may also comprise a second internal catheter tube (which may alternatively serve for inflation of the balloon) or other element that extends through the tube 3 to within the balloon 2, as indicated at 4 in phantom outline in FIG. 8, to assist in locating the tip of the balloon in the desired position. FIG. 8 shows the GI mapping catheter positioned in the gastric antrum indicated at G and before inflation, and FIG. 9 shows the catheter after inflation to cause multiple electrodes 1 to contact the mucosal surface around the interior of and spaced lengthwise of the GI tract, sufficient to obtain electrical potentials indicative of GI electrical activity around and lengthwise of that part of the tract. The electrodes are preferably but not exclusively point electrodes, such as convex pointing electrodes, which at least when the balloon 2 is inflated stand perpendicular to the surface of the balloon, such that they indent the mucosa to enhance contact and signal quality.

An alternative form of GI mapping catheter may comprise an expandable mesh, carrying a similar array of spaced electrodes, and formed of a resilient plastics material or a spring metal such as surgical grade stainless steel, and having a memory for its expanded position, which is mechanically restrained unexpanded until in position within the GI tract.

For example an electrode array of a GI mapping catheter of the invention may comprise between 1 and 10 rows of electrodes spaced lengthwise of the catheter between the proximal end (coupled to tube 3) and the distal end, each row comprising between 3 and 20 electrodes spaced around the catheter, providing an array of between 3 and 200 electrodes for example. In an alternative embodiment the electrodes 1 may be arranged in rows angled or tangential to the longitudinal axis of the catheter, with, when the catheter is an expanding mesh catheter, an electrode at each or at least many intersections of mesh elements, over a part of the major surface area of the mesh catheter.

In relation to the electrode form, desired qualities for GI electrical signals acquired by the electrodes are an adequate signal to noise ratio (SNR) (the gastric mucosa has high impedance and attenuates signal), a stable baseline, and preferably a steep negative descent at the down-slope of the slow wave signal. As stated the electrodes are preferably protruding, to press into or indent the mucosa to achieve an adequate SNR. Smaller electrode diameters will generally achieve a steeper down-slope (shorter duration of activation over the electrode signal; quicker offset to onset period). However, if the electrodes are too protruding and of too small a diameter, they may puncture the gastric mucosa rather than press into it. A suitable form electrode may comprise a conductive protrusion of between 2 and 5 mm, or 2 and 3 mm, or about 2.5 mm in length (from the electrode carrier or electrode base to the tip of the electrode), and of a cross-sectional dimension (such as diameter if the electrodes have a circular or similar cross-section) of between 0.3 and 3 mm, or 0.5 and 1.5 mm, or 0.7 and 1 mm, or about 0.8 mm. The electrodes may suitably comprise sintered Ag-AgCl electrodes.

The catheter has been described above in relation to, and as suitable for, insertion through a natural orifice into the GI tract but in an alternative embodiment one or more rows of electrodes may be carried on another form of electrode carrier such as an element for example a flexible pad, adapted to contact the external serosal surface of the stomach. Such an electrode carrier may be surgically inserted for example via laproscopic or keyhole surgery into the abdomen and positioned against the exterior of the stomach. An example is shown in FIG. 9a in which 90 indicates a flexible electrode pad which when tightly furled or rolled is inserted through a keyhole incision made in the abdominal wall. Laproscopic graspers 91 are used to unfurl the electrode pad and position it against the external surface of the GI tract as shown, such that electrodes 1 carried by the pad 2 contact the serosal surface. The electrodes are connected via a cable 93 which passes back out through the abdominal wall to instrumentation. After mapping, the electrode pad 90 is re-furled and removed back through one of the incisions in the abdominal wall.

### GI Mapping System and Method

In use a GI mapping catheter as described is connected by a cable to a signal acquisition stage of a GI electrical activity mapping system of the invention and once the GI catheter is positioned by the clinician in the GI tract, and engaged with the mucosal wall, the clinician may activate signal acquisition, typically via a graphical user interface. The GI mapping system is arranged to receive and process multi-channel electrical signals from the mapping catheter electrodes 1, either all or at least those making good contact, and is arranged to identify GI slow waves and spatially map the GI myenteric electrical activity (herein referred to as GI smooth muscle or slow wave electrical activity) preferably in real time or near-real time, and identify re-entrant GI electrical loops. The system may typically comprise a computer including a processor, program memory, and an operator interface including display or VDU which may be a touch-input screen and optionally also a keyboard or keypad, and a communications interface, coupled by a data bus.

The analysis processing by the GI mapping system of the electrical potential signals detected at the electrodes includes identifying GI electrical slow waves and mapping the electrical activity, which may include producing any one or more of an activation time map or maps of gastric electrical waves or wavefronts, a velocity field map or maps, an amplitude map or maps, all either as pixelated or isochronal maps or in other form, and which may also or alternatively animate any one or more of the same and/or GI slow wave propagation generally. The analysis processing may include mapping and/or animating the GI electrical activity or propagating waves over a generic or subject-specific anatomical model, running on the system processor. The GI mapping system is arranged to carry out analysis to identify re-entrant GI electrical loops. This analysis processing may also include comparing the mapped GI electrical activity to a stored reference database to provide an indication of normal or abnormal GI electrical activity.

FIG. 10 shows an example of a user-display on a VDU 20 that a GI mapping system may present to a clinician during an examination. On the upper right indicated at 21 is a live video-endoscopy view of the gastrointestinal tract lumen. On the upper left indicated at 22 is a view of a generic or optionally subject-specific anatomical computer model of the section of the GI tract, over which the GI electrical activity or slow wave information obtained from the electrode array is mapped and may be animated and from which a clinician may determine re-entrant GI electrical loops or on which the system may highlight to the clinician any re-entrant GI electrical loops identified by the intelligent system. The live electrical potentials from a selection of channels from the electrode array are shown at 23. The system may be arranged to determine or approximate the relative locations of the electrodes in contact with the interior surface of the GI tract, to register same correctly to the model and optionally to develop or modify the model. The system may be arranged to display gastroscopic view 21 initially full screen, and after the mapping catheter is inserted and expanded the gastroscopic view may be reduced to the window 70 or closed, the electrophysiological recordings, and mapped electrophysiological data such as activation time map(s), velocity map(s), amplitude map(s), dysrhythmia map(s), and/or other wavefront propagation displayed as 2D or 3D images and/or animations shown in real-time. The system of the invention may also be arranged to record the session or to communicate the GI electrical data to another system for offline or further analysis and/or storage.

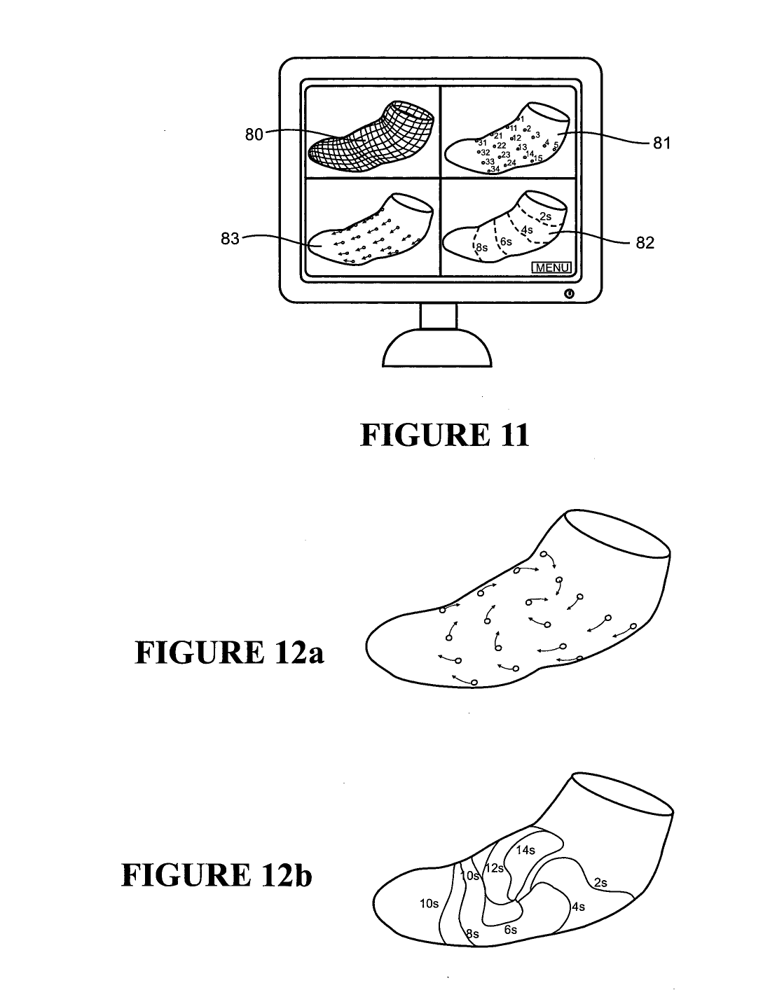

FIG. 11 shows another example of or an additionally available user display of a GI mapping system of the invention. A representation of an anatomical model of a stomach shape (or part thereof) is indicated at 31. The position of the electrodes of the array on the model (for example, for selecting channels to view) is indicated at 32. The electrode positions may be numbered. An activation time map which comprises isochronal propagation of GI slow waves on the stomach model is indicated at 33. An isochronal map comprises a two-dimensional contour plot showing the spatiotemporal sequence of GI slow wave activation. A velocity map which comprises multiple individual vectors on the model indicates the velocity and direction of GI slow wave propagation at each electrode is indicated on the model at 34. A clinician may identify re-entrant GI electrical loops from any one or more of these displayed maps or the system may highlight to the clinician on any of these maps any re-entrant GI electrical loops identified by the intelligent system. The system may be arranged to produce and display and optionally animate on a model in 3D the GT electrical activity map(s).

In FIG. 11, in the activation time map and velocity map at windows 33 and 34 the gastric electrical activity is shown propagating normally. FIGS. 12a and 12b show respectively similar activation time and velocity maps in which in contrast a GI slow wave is looping and propagating abnormally.

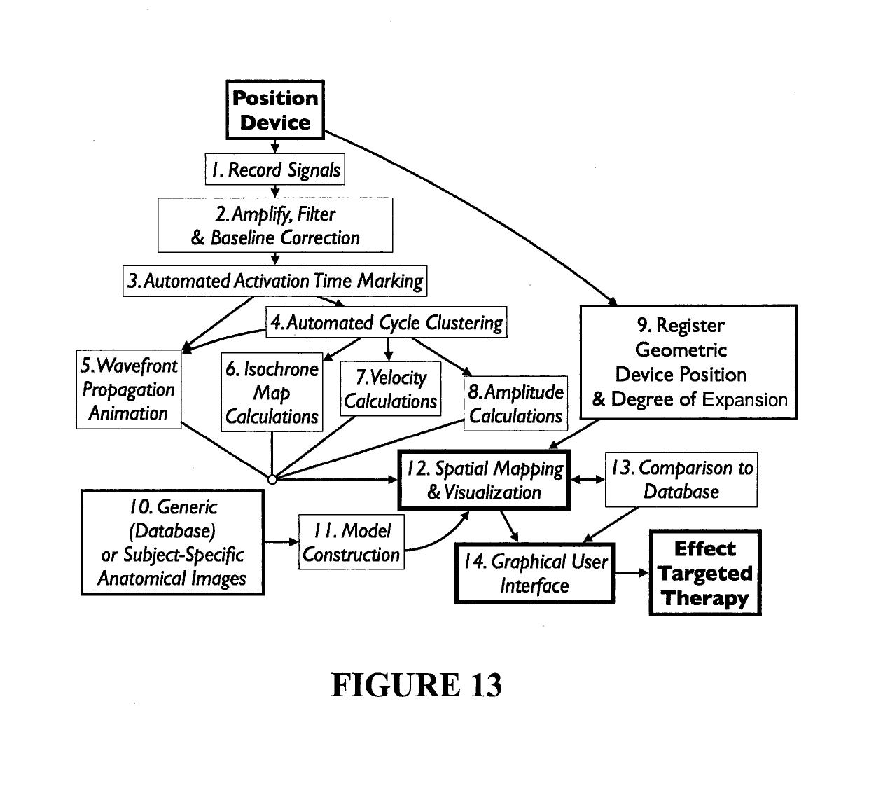

FIG. 13 is a flow chart illustrating signal analysis, mapping, and model fitting stages of a preferred embodiment of the invention. The darkest outline boxes indicate key user inputs, medium outline boxes indicate key integrated outputs, and lightest outline boxies indicate computer processing steps. After positioning a GI catheter and recording or beginning to record electrical signals from the electrodes, and any amplifying, filtering, and baseline correction, GI electrical slow wave events at electrodes are marked, and clustered or partitioned into clusters of electrical events each relating to a discrete GI electrical slow wave cycle. One or more of velocity calculations, amplitude calculations, and isochrone map calculations are performed by the system processor. The resulting activation time, velocity, and amplitude information may then be spatially mapped in 2D or 3D in pixelated or isochronal or other form, optionally on a generic or subject-specific computer model of the GI tract or the part thereof. The model may be a stored generic model or one of a number of stored generic models of the GI tract or a part thereof, or may be constructed from a subject's specific anatomical images of the GI tract acquired prior to the EGG examination, for example via MRI or CT scanning. The catheter position and degree of expansion and thus individual electrode positions are registered on the map or model and the velocity, amplitude, and/or isochrone data fitted to the map or model, and displayed to the clinician on a VDU as 2D or 3D maps or animations. A wavefront propagation animation may be produced from the marked or marked and clustered GI slow wave events and also displayed. A clinician may identify re-entrant GI electrical loops from any one or more of these displayed maps or the system may be arranged to compare the mapped GI electrical activity to a database and identify and highlight re-entrant GI electrical loops. and a clinician may interface with the system via a touch screen, keypad, computer mouse or similar through an appropriate menu or non-menu based interface system. The clinician may use the resulting analysis to effect targeted therapy for the patient.

Many of individual system blocks of the preferred embodiment system of FIG. 13 are now described in further detail.

### Signal Recording

Signal acquisition may for example be at a sampling resolution of >1 Hz, typically at ˜30 Hz, and up to 512 Hz or greater. In a signal acquisition stage the signal channels may be digitized and amplified, and filtered to remove low frequency drift and wandering baselines, important for mucosally-acquired low amplitude and low frequency GI electrical signals, and to remove unwanted artifacts and noise.

### Automated Activation Time Marking

“Activation” as used herein refers to a rhythmic spontaneous inward current in interstitial cells of Cajal, causing the cell membrane potential to rapidly rise. In extracellular recordings the onset of this depolarization termed “activation time” or AT signals the arrival of a propagating electrical wavefront to a particular location in the tissue. ATs must be identified (“marked”) at each electrode site. The marked electrode ATs are used to generate an activation time map or maps which provide(s) detailed spatiotemporal visualization of the spread of GT electrical activity across an area of tissue. ATs are identified to produce an activation time map or animation.

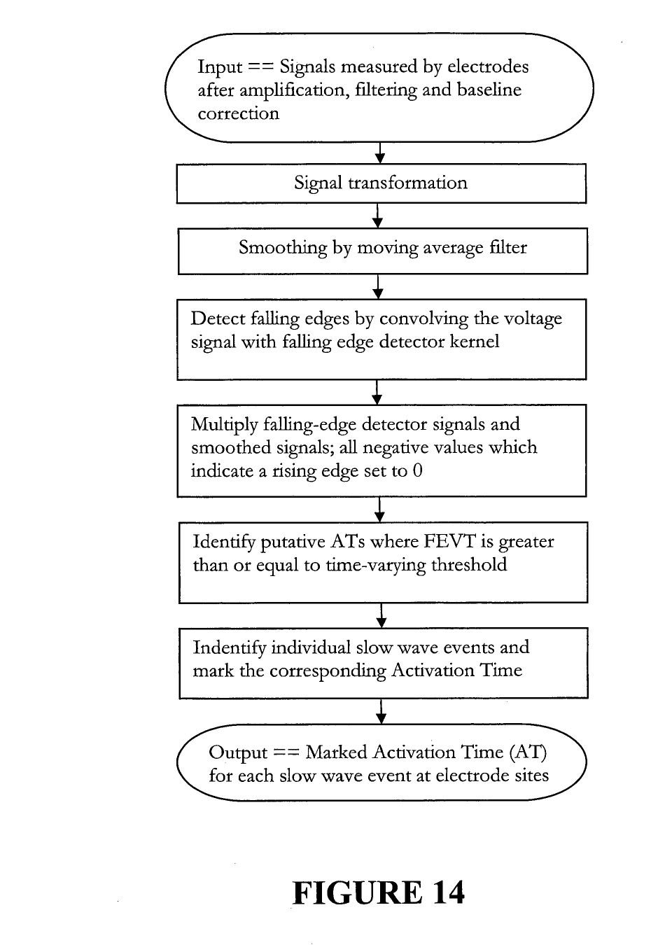

A preferred method for automated AT marking is a falling edge varying threshold method, which comprises transformation, smoothing, negative edge detection, time-varying threshold detection, and AT marking of the signal from each electrode. FIG. 14 is a flow chart of a preferred embodiment of an FEVT method for GI slow wave activation time identification.

Transformation can be carried out by for example negative derivative, amplitude sensitive differentiator transformation, non-linear energy operator transformation, or fourth-order differential energy operator transformation. A moving average filter of a tuneable width is applied to the transformed signal to smooth the signal. The transformation amplifies the relatively large amplitude, high frequency components in the recorded signal, which corresponds to the onset of activation. Subsequent filtering increases the SNR of the transformation by reducing high frequency noise.

An edge detector kernel is then be used to identify falling edges within the smoothed signal. A falling edge produces a positive deflection in the signal from the edge detector kernel, and a rising edge produces a negative deflection.

A FEVT signal is then calculated by multiplying the signal from a falling-edge detector and the smoothed signal, and then all negative values which indicate a rising edge are set to 0.

In the preferred form a time-varying threshold is calculated from the FEVT output, by computing the median of the absolute deviation in a moving window of predefined width. The centre of the moving window consecutively shifts one sample forward, such that the threshold is computed for each point in time over the duration signal. Such a variable threshold improves detection accuracy by accounting for slight deviations in the waveforms of recorded signals. A constant threshold may be used but a time-varying threshold may reduce potential double counting and mis-marking. Signal values greater than or equal to the threshold define the times at which slow wave events might occur.

Individual slow wave events are then identified from the resulting data set which may contain multiple slow wave events, by imposing a criterion that distinct events must be separated by a minimum time.

### Automated GI Slow Wave Cycle Clustering

The ATs as are clustered based on temporal closeness, into distinct cycles that partition the discrete propagating GI slow wave wavefronts. Clustering identifies individual GI slow waves based on a temporal closeness criterion, and proceeds in iterative fashion. Consecutive members in a data set are grouped as representing the same GI slow wave event if they are close enough in time to an estimated activation time. Such estimation employs deriving the best-fit second order polynomial surface, based on the location of electrode sites and the activation times detected at them. The estimated activation time is computed by extending said polynomial surface to the candidate location for clustering. The maximum time difference allowed to cluster two members is termed the time tolerance; its value must be long enough to accommodate small estimation errors and identify fractionated waveforms as single events, but short enough to properly partition distinct GI slow waves. When no more members of the data set meet this closeness criterion, a new cluster is formed to represent the next GI slow wave event. Auto clustering groups all marked data into individual clusters, each delimiting an independent GI slow wave event

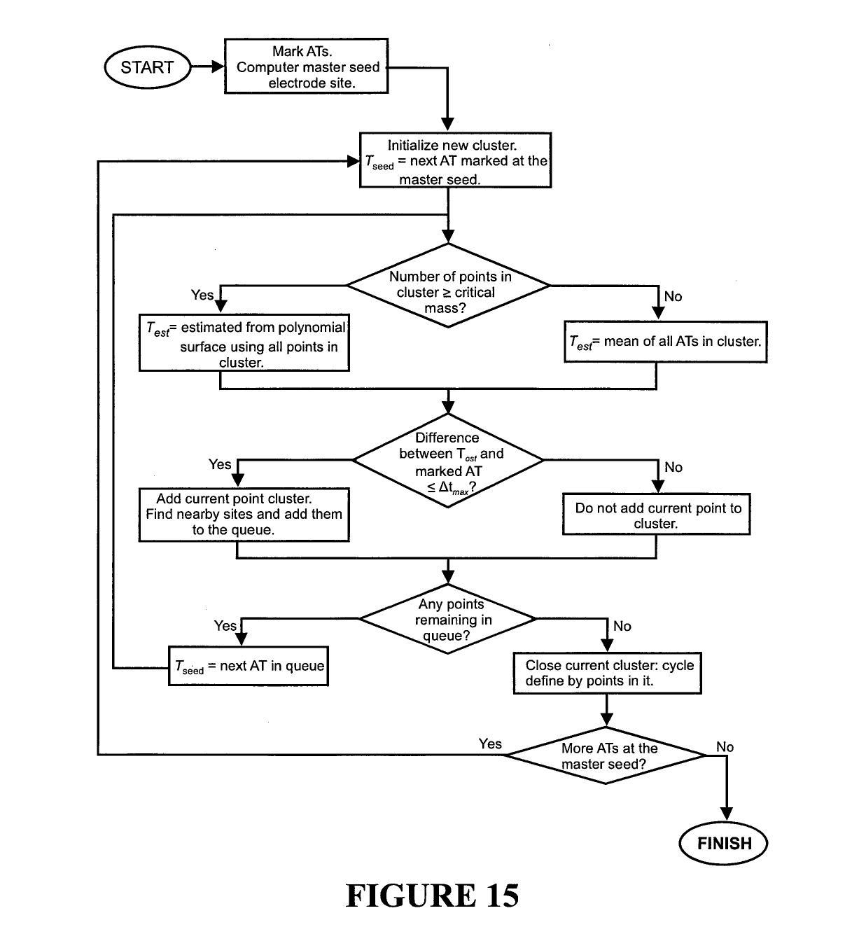

FIG. 15 is a flow chart of a preferred embodiment clustering method termed region growing using polynomial surface estimate stabilization (REGROUPS) for clustering (x, y, t) points representing ATs into groups representing independent GI slow wave cycles, where (x, y) denotes the position of an electrode site and t denotes an AT marked at that site, and t denotes the activation times identified at that site.

The algorithm is initialized by automatically selecting a “master seed”, which is an electrode position embedded in a region with the maximal density of information about a propagating wavefront. The cluster is then grown outward from the region where the spatial density of data is highest, ensuring that the subset of points initially assigned to the cluster is statistically cohesive and limiting the possibilities of assigning noise signals to a nascent clusters. The master seed may be selected by first calculating the total number of ATs detected at each electrode site, then finding the centre of mass and selecting the seed location as the electrode closest to the centre of mass. Once the master seed is located, a queue containing the nearby electrode sites' ATs in a specified circular range of the master seed is created and the first AT in the queue becomes the current seed. Each AT is tested for membership of a cluster based on comparison to an estimated AT, which is derived by fitting (in the least squares sense) a second-order polynomial surface to the data points already assigned to the cluster. The 2^{nd} order surface acts as a continuously updating spatiotemporal filter: if the time difference of estimated AT and tested AT is small enough, then the tested AT is considered as representing a same wavefront as the seed and is assigned to the cluster. Once assigned, the point is not assessed again. If the tested point is clustered, all of its neighbour electrodes and marked ATs at these electrodes are added to the back of the queue, providing they are not already in it. If a tested point is not clustered, it may be tested again for membership only after a new cluster is initialized at the next iteration. This restriction forces all wavefronts to be independent. Regardless of whether any point is added to the cluster, the current seed is removed from the queue and the next electrode site becomes the current seed. Thus, the region in (x, y, t) space representing an independent cycle grows, and terminates when the queue of nearby points becomes empty. At this stage, the cluster contains all ATs from one GI slow wave cycle. The same process is repeated to identify another independent cycle, starting with the next sequential AT marked at the master seed. Each iteration produces a cluster of (x, y, t) points which represent the dynamics of an independent GI slow wave cycle, from which wave front propagation, an actuation time map may be produced, and isochrones map calculation, velocity and amplitude calculation can all be realized.

### Activation Time or Isochronal Mapping

An activation time or isochronal map comprises a contour plot of GI slow wave activation. An isochronal map may comprise a spatial representation of the electrode sites, and the isochrones (contour lines), which represent the spatial distribution of ATs lying within the same specified time window, i.e. sites with similar activation times. In a preferred form the temporal resolution (i.e. isochrone interval) may be about 0.5 seconds when the activity is fast (>10 mm/s), about 2 seconds when the activity is slow (<4 mm/s), and about 1 second when the activity is from 4-10 mm/s, for example. Information such as speed and direction of propagation may be inferred from an isochronal map.

The spatial interval of two neighboring isochrones can be used to calculate the velocity of slow wave propagation.

An activation time or isochronal map may be produced by:
Plotting the identified ATs in the same spatial arrangement as the electrodes.
Mapping the ATs to the electrodes to which they pertain, in the same configuration as the electrode matrix. The value of each AT may be represented by a colour or colour tone in a colour or colour tone spectrum that represents the appropriate range for the activation values. A look up ‘configuration file’ may contain information on electrode distribution and inter-electrode distance; the electrode numbers may be stored in a matrix, with the corresponding electrode number reference by the indices.

A pixelated isochronal map may be converted into a smooth, filled contour map with isochronal lines spaced at a specified time interval.

Poor electrode contact to the mucosal surface may result in areas with imperfect electrical recordings. To represent the entire activation field, areas with bad contact may be interpolated based on the surrounding ATs. Inactive electrode sites surrounded by several active sites are preferably interpolated into the AT map. In a preferred form a 2-stage spatial interpolation and visualization scheme may conservatively interpolate inactive electrodes using information from neighboring active electrodes on the basis that if an inactive electrode site is bordered by three directly adjacent (including diagonal) active electrodes, the AT is linearly interpolated from adjacent active sites' ATs, and correspondingly pseudo-colored (an “interpolated site”). If the total number of active plus interpolated sites bordering a still-blank site is four, then the still-blank site in interpolated. Such a 2-stage scheme, as opposed to a recursive one, prevents a run-away interpolation process from inappropriately filling in blank sites across the entire array.

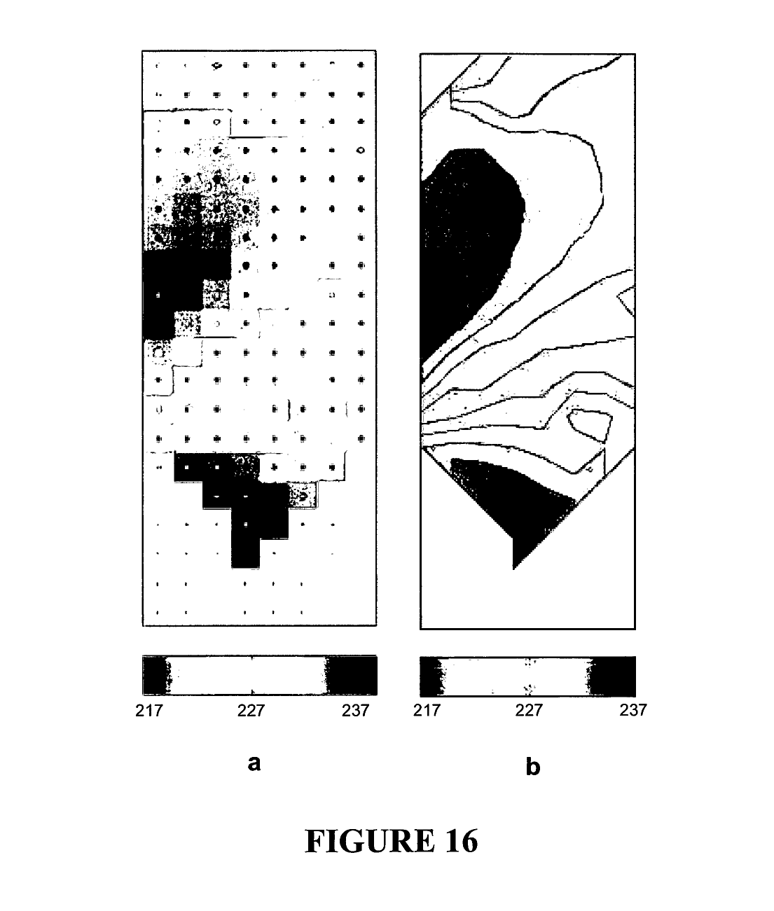

FIG. 16a shows a pixelated isochronal map or a part thereof and FIG. 16b shows such a smooth filled contour map with isochronal lines. In FIG. 16a black dots indicate electrode sites at which an AT was marked, and white dots indicate electrode sites for which no AT was marked, but in some cases was interpolated. The ATs are color coded to propagate from for example red to blue, representing the earliest and latest ATs respectively over a 20 second interval from second 217 to second 237. In FIG. 16b the isochronal lines are spaced at 2 second intervals.

An isochronal map may also be applied over an anatomical geometry model in 2D or 3D to aid visualization and accurate diagnosis for the clinician.

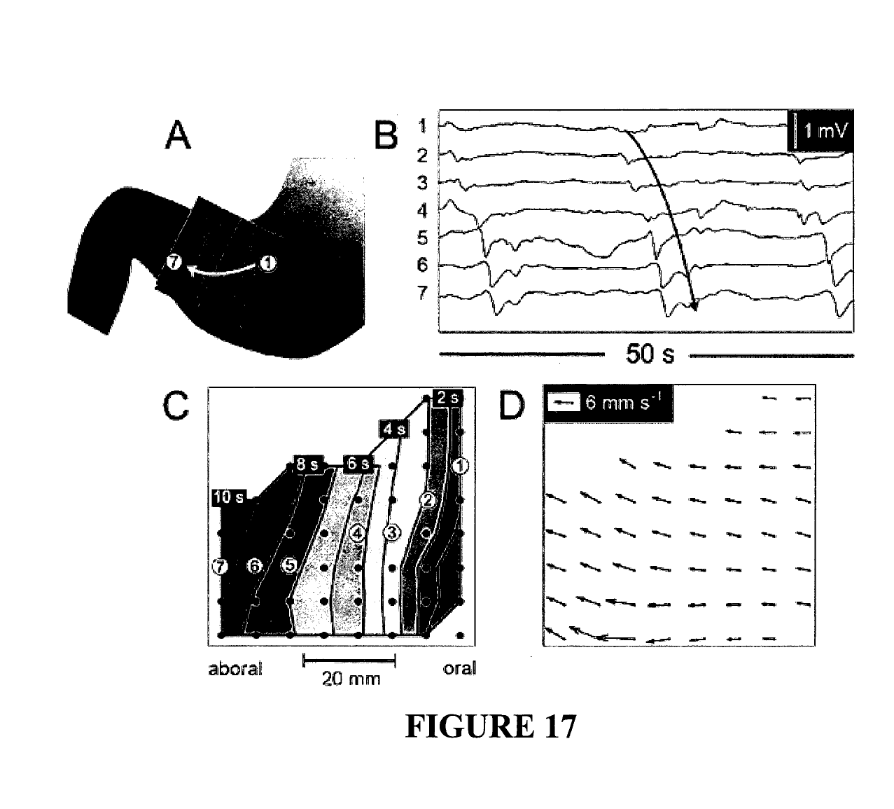

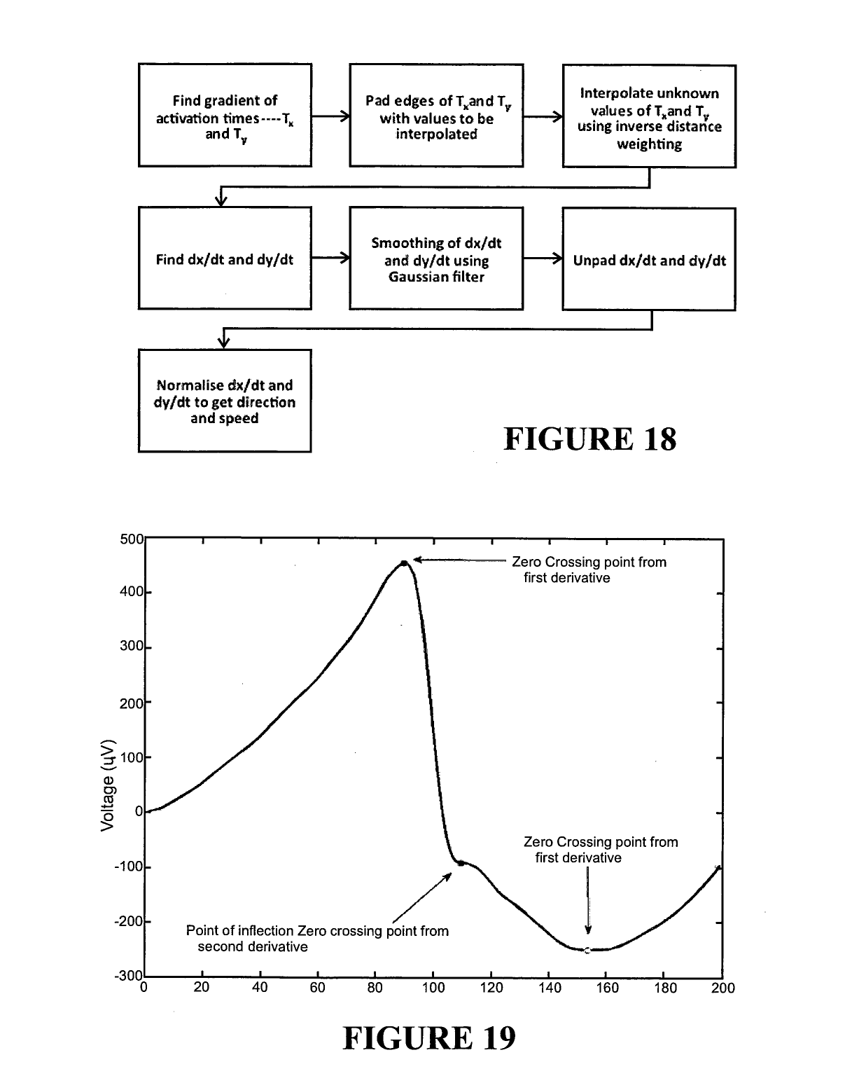

A velocity field may be mapped in 2D or displayed over anatomical organ geometry in 3D in a similar way to as described for activation time mapping. FIG. 17a is an isochronal activation time map, and FIG. 17d is a calculated velocity field map.

### Wavefront Propagation Animation

The wavefront propagation may be directly animated from the ATs, or clustered ATs to provide animations of an improved accuracy or clearer visualization to convey information of a propagation wave behaviour, including complex behaviors such as occur in slow wave dysrhythmias. Separate colors may be assigned to the discrete wavefronts in the animations (or map(s)). In one embodiment, animation may be performed by:
Configuring a computational array in the same configuration as the recording electrodes array.
Checking each location on the recording electrode array at each specified time frame (for example at 1 second intervals), and if an AT occurred at that electrode within that time frame, then representing the pointer pixel in the computational array corresponding to the location of the electrode highlighted or in a different colour than those electrodes at rest.
Causing the thus ‘activated’ electrode(s) to stay highlighted or coloured for a fixed duration before turning off again (i.e. going back to ‘rest’). The highlighted or coloured point may fade as it disappears.
Different colours may be assigned to distinct clusters each relating to a discrete GI electrical wave, for example based on a repeating pattern of a few colours.

Animation(s) may also be on an anatomical geometry model to aid visualization and accurate diagnosis for the clinician as will be further described. Preferably the animation(s) may be zoomed and rotated.

### Velocity Calculations and Mapping

GI slow wave propagation velocity in the stomach varies. Differences may be greater during dysrhythmia. Velocity calculations may assist in diagnosing at least some dysrhythmias. The change in wavefront orientation and the onset of anisotropy are important clues to the diagnosis. The velocity map can be simply obtained from gradient field of the activation times. Preferably interpolation and smooth filter are also applied to obtain a more accurate and smooth velocity field.

The activation times are define as T(x,y) in an two dimensional array and the velocity field is defined as in Equation 1. Each of the velocity field vectors represents the direction of wave front propagation and the speed at which the wave is travelling at that time instant.

$$V ( x , y ) = [ \begin{matrix}\frac{d x}{d T} \\ \\ \frac{d y}{d T}\end{matrix} ] = [ \begin{matrix}\frac{{T}_{x}}{{T}_{x} + {T}_{y}} \\ \\ \frac{{T}_{y}}{{T}_{x} + {T}_{y}}\end{matrix} ] \\ {T}_{x} = \frac{\partial T}{\partial x} \text{:} {T}_{y} = \frac{\partial T}{\partial y} \qquad Equation 1$$

The gradient of the activation times Tₓ and T_{y} could be calculated via a finite difference approach. The finite difference approach uses centred difference in the internal 2D array of the activation times and uses two point one sided differences on the edges of the 2D array of activation times.

A smoothing filter may be introduced to reduce edge effects, and unknown values in the gradient array need to be obtained by interpolation, preferably using an inverse distance weighting method as shown in Equation 3.

$$Y = \frac{{\sum }_{i = 1}^{n} X / {D}^{2}}{{\sum }_{i = 1}^{n} 1 / {D}^{2}} , \qquad Equation 3$$

where Y is the unknown value to be interpolated and the X is the known value, and D is the distance between the known (X) and unknown values (Y).

Once the gradient array of the activation times is obtained, the velocity field vectors can be calculated from the gradient field vectors using Equation 1 above.

A smoothing function is then applied to the velocity field vectors to reduce noise artefacts. Preferably the smoothing filter is a Gaussian filter, where the output is a centrally averaged weighted value, for example Equation 2 below shows a Gaussian Filter which could be used.

$$Gaussian Filter coefficients \\ F ( x , y ) = \frac{{F}_{n} ( x , y )}{{\sum }_{x} {\sum }_{y} {F}_{n}} , where {F}_{n} ( x , y ) = {e}^{\frac{- ( {x}^{2} + {y}^{2} )}{2 {σ}^{2}}} , x = 5 , y = 5 , σ = 0.75 \qquad Equation 2$$

After the smoothing function, the interpolated values are removed from the array. Finally the velocity array is normalized to retrieve direction and speed information from each vector.

### Amplitude Calculation and Mapping

Extracellularly-recorded slow wave amplitudes may be indicative of pathology and/or dysrhythmia because amplitudes may be low in some diseases, where interstitial cell of Cajal networks are degraded and/or dysrhythmia may be associated with regional high or low slow wave amplitudes. A slow wave amplitude may be calculated based on the identified AT of an event.

The amplitude may be calculated by using a fixed time window and identify amplitude as the differences between peaks and troughs for event falls in the window. However, such method has potential inaccuracies. FIG. 19 illustrates identification of a peak and two troughs of a single event in a GEA trace. The main steep signal event is followed by a slow varying signal back to baseline. This slow variation could be noise artefact, and the first trough point (red) is the correct point for amplitude determination, rather than the second trough point (green).

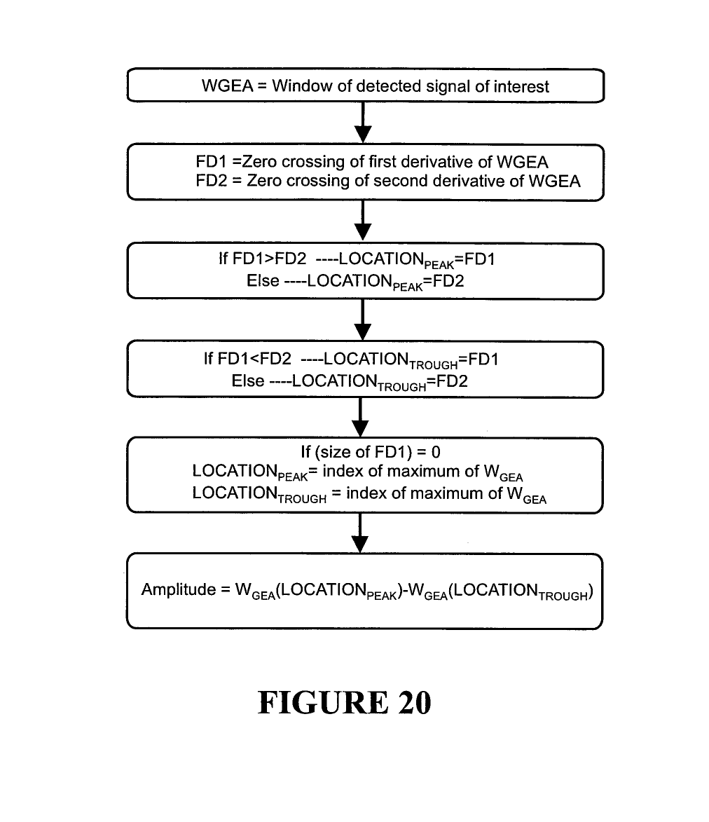

Referring to FIG. 20, in order to automatically and accurately identify correct peaks and troughs in problematic signals, a preferred method uses the ‘zero crossing’ of the first and second derivative of the signal. A flow chart of the zero crossing method is shown in FIG. 20.

First of all a fiducal point is chosen as a detected gastric event (GEA) within detected signals, a fixed window is then applied to select signals centred around the fiducal point. The amplitude signals which fall into this window of selection are subject to first and second derivative calculation and zero crossings of the first and second derivative are located. A zero crossing of the first derivative indicates either a peak or trough has occurred at that time instant, whereas a zero crossing from the second derivative indicates a point of inflection. A point of inflection is where the original signal changes its sign of curvature, for example from negative curvature to positive curvature, or from concave downwards to concave upwards. For example in FIG. 19, there are two zero crossings a and c for the first derivative and one zero crossing b for the second derivative. The signal value at every zero crossing is then fetched for comparison to determine whether a peak or a trough has occurred at these zero crossings. All zero crossings before the fiducal point are considered to be possible peak locations and all zero crossings after the fiducal point are considered to be possible trough locations. Next the method compares signal values at zero crossings of first and second derivative before the fiducal point to determine a peak location. The first sought after location just before the fiducal point is chosen as the peak. Similarly the method then compares signal values at zero crossings after the fiducal point to determine a trough location. The first sought after location just after the fiducal point is chosen as the trough. The amplitude can then be calculated using chosen peak and trough signal values. If there is no zero crossing found for the first derivative within the time interval, which indicates that the signal is constantly increasing or decreasing within the defined time interval, then the amplitude is the difference between the maximum indexed signal value and the minimum indexed signal value.

### Spatial Visualization of Velocity and Amplitude Profiles

The spatial representation of the electrodes may be defined in a system ‘configuration file’ (also used for the activation time maps), which includes information on the inter-electrode distance.

Velocities are calculated as described above from the activation time map values. The propagation speeds at each active electrode are assigned colours from a spectrum range, and are then displayed as a ‘speed map’ according to the configuration file. Arrows representing normalised velocity vectors are then overlaid on the speed map to create the ‘velocity map’.

Amplitude values are calculated for each wavefront as described above. These values are then assigned a colour from a spectrum range, and displayed as an ‘amplitude map’ according to the configuration file. The colour range assigned to the amplitude and speed maps is then interpolated to give smooth colour transitions or ‘contour maps’, that allow for easier visualization and interpretation.

### Registration of Device Position and Expansion

The electrode array position may be anatomically registered in the GI tract by for example:
The system may be arranged to display the position of the mapping catheter in a model stomach geometry which in conjunction with a displayed an endoscopic view assists the clinician to position the catheter where desired.
By a second roving anatomical catheter arranged to a low-current locator signal to a reference electrode, measuring and transmitting samples, against a 3D referencing system, for the construction of a geometric matrix or ‘virtual lumen’. The position of the mapping catheter and electrode array is also registered within this matrix by the 2^{nd} catheter.
By imaging e.g., plain film radiography in 2 axes, and then forming a mesh based on the identified electrode positions.

In one embodiment a measuring system is arranged to measure the volume of air or other fluid installed into an inflatable mapping catheter via a syringe or pump. The user instills a sufficient volume until the electrodes press against the gastrointestinal tract mucosa. Air may also be removed from the tract, via endoscopic suction, such that the tract walls collapse down around the device. The degree of inflation determines the final spacing of the electrode array because the electrodes move further apart during inflation. In a preferred embodiment the electrode spacing at the time of mapping is determined by:
The value of air of liquid instilled is measured, for example visually identified by a volume scale on the syringe or other device used to effect the inflation.
This volume is input by the user into the system.
The post-inflation surface area of the device is calculated by the system.
The spacing of the electrodes at the time of mapping is calculated by geometric calculations that define the distance between points on a 3-dimensional surface, with these distances being proportional to the degree of inflation.

The calculated ‘inter-electrode distance’ on the expanded device, at the time of mapping, is subsequently used by the system in calculating the activation times, clustering, isochrone, velocity, and amplitude mapping and animations.

Model Selection from Generic Database, or Subject-Specific Model Development

A subject-specific anatomical model of the mapped part of the GI tract may be produced by for example:
A medical image or image set providing a 21) or 3D description of an organ position is obtained, for example via ultrasound, MRI, CT, or plain abdominal x-ray of the patient.
The GI tract section of interest is extracted via manual (tracing the organ outline) or automated (determined by imaging density transition zones) segmentation methods to create a 3D data cloud representing the surface of the GI tract section.
A finite element mesh is created to match these data points using a non-linear iterative fitting method.

The system may comprise a database of multiple models along with corresponding data on how each was acquired e.g. sex, age, imaging methodology, medical history, pathological conditions, and an appropriate model may be recalled from the database by the system based on data such as demographic data relating to the patient entered by the clinician, for example the patients' sex and age data. For example, if a 5 year old female child is being examined, a mean stomach geometry for five-year old female children can be automatically presented to the clinician. Alternatively, a library of models may be stored for review by the clinician, to manually select one that best matching the stomach geometry of the patient under examination. This library is arranged in size order for intuitive browsing.

### Model Construction and Mapping to Model

Construction of a specific anatomical model brings together:
registration of the mapping catheter position and degree of expansion, and
the anatomical stomach geometry model chosen by the clinician
   to create a model specific for the GI tract section and patient under evaluation. The chosen anatomical geometry model is reconfigured to match the calculated geometry resulting from the mapping catheter expansion, for example by:
The calculated geometry of the expanded electrode array geometry is used as the ‘true’ reference geometry, being empirically determined at the time of the procedure.
The reference model geometry is resized by geometrically expanding or reducing the model proportions until they match the ‘true’ reference geometry proportions at the position of the mapping catheter within the GI tract.

With a specific model that best represents the anatomy under evaluation, and the position and degree of expansion of the mapping catheter and electrode array, 2D or 3D activation time, velocity, and amplitude maps and animations may be applied to the model and displayed as referred to previously. For example this may be achieved by:
Common landmark points on the model and the locations of the recordings relative to these landmark points are identified in the model.
The root mean squared distances between these common points are minimized.
Activation time, velocity and amplitude maps are “texture mapped” or orthogonally projected onto the surface of the model.
Results from multiple recording sites can be combined to enable results from different regions to be compared in the relative locations at which they were recorded.

### Analysis Comparison to Database

The system and method of the invention may facilitate an accurate diagnosis by allowing the clinician to compare the mapped GI slow wave data to standard reference (normal population) data, or the system may be arranged to identify and highlight re-entrant GI electrical loops. A specific diagnosis may be automatically suggested by the system, based on characteristic differences from the normal population.

In a user menu in the system interface, the clinician may select to review slow wave amplitudes for a specific time period of the recording. The system is arranged to present a comparison to a standard reference range.

As a further example, to detect re-entrant loops, activation times of individual slow wave cycles are identified and isochronal activation maps and velocity maps are calculated for every wave cycle. In a user menu in the software interface, the clinician may select to review slow wave propagation and velocity for a specific time period of the recording i.e. specific slow wave cycles occurring during that period. As well as spatially mapping the isochronal activation patterns and velocities for the selected time period, the system is arranged to perform the following steps to present a comparison to the standard reference range:
Average the velocities of each cycle to calculate a statistical mean velocity and standard deviation for each cycle, and preferably separate the longitudinal and circumferential velocity components.
Average velocities across all cycles are calculated to generate a mean and standard error of the mean for the total velocity, and the total longitudinal and circumferential velocities.
The resultant values are statistically compared to a standard reference database.
The result is displayed in the software interface. For example, if the circumferential components of the slow wave velocities of a patient with functional dyspepsia are statistically found to be higher than that of the standard reference range (i.e. ˜zero mm/s circumferential propagation in the normal human antrum, then a display item indicates this. The clinician may note the finding, and conclude that an antral dysrhythmia is occurring, contributing to a diagnosis.

The clinician may then institute a targeted therapy into the location where the dysrhythrnia is occurring, such as pharmaceutical agent, or pacing or ablation therapy, to interrupt the dysrhythmic mechanism. The targeting of this therapy can be specifically guided by the anatomically visualized spatially represented isochronal slow wave maps, or animations, to ensure it is accurately delivered.

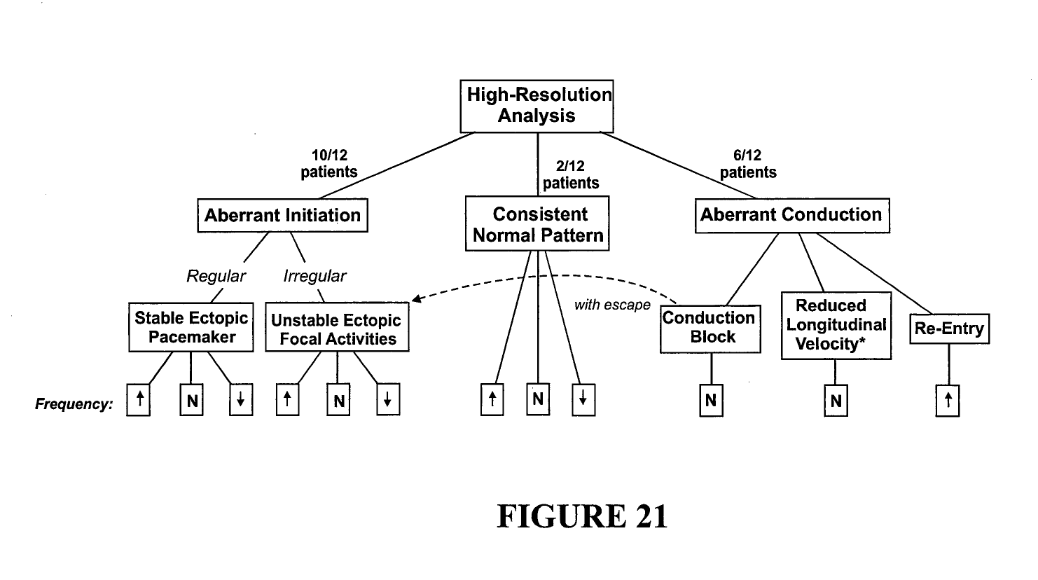

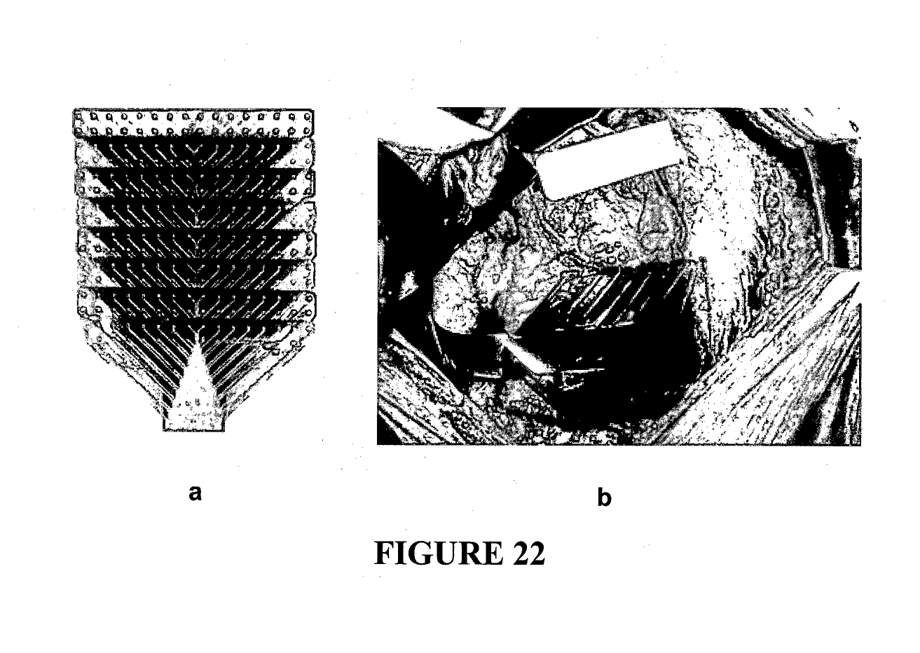

FIG. 21 is a flow chart of an embodiment of a spatial classification scheme for slow wave abnormalities in gastroparesis, based on High-Resolution serosal mapping in a cohort of 12 patients with diabetic (n=8) and idiopathic (n−4) disease. Observation of rapid circumferential conduction, associated with high amplitude extracellular signals, is shown to be of novel diagnostic value in: stable ectopic pacemaker, unstable ectopic focal activities, conduction block (incomplete, or with escape), and re-entrant activities.

### EXAMPLES

The invention is further illustrated, by way of example and without intending to be limiting, by the following description of trials work.

### Example 1

Patients with diabetic gastroparesis were recruited and consented. Each patient had documented delayed gastric emptying on a 4-hr standardised scintigraphy study, to at least 20% gastric retention at 4 hrs. The mean 4-hr gastric retention of this group was 29%. The mean symptom score (on a 20-pt standardized scale) was 16/20.

### Methods of Mapping

Flexible printed circuit board (PCB) multi-electrode arrays consisted of copper wires and silver or gold contacts on a polyimide ribbon base (‘PCB electrode array’). The recording head of each individual PCB—electrode array had 32 electrodes in a 16×2 configuration, with an interelectrode distance of 4 mm. In each experiment, 7-8 PCB—electrode arrays (224-256 electrodes total; area 36 cm²) were arranged in a square configuration (see below) to map ˜⅓ of the anterior gastric surface with each placement.

Mapping was undertaken immediately after opening the abdomen and prior to manipulating the organs or commencing any surgical dissection. The PCB—electrode arrays were laid directly on the anterior surface of the stomach; the posterior gastric surface has not yet been mapped. Once placed, the locations of the PCB—electrode arrays was defined with reference to several anatomical landmarks: the gastroesophageal junction (defined by the angle of His), the apex of the fundus, the junction between the corpus and antrum (defined by the nerves of Latarjet) and the pylorus (defined by the vein of Mayo). Warm moist gauze packs were laid on top of the PCB—electrode arrays to ensure that gastric contact was maintained. Care was taken to allow the PCB—electrode arrays to move freely with the respiratory excursion, and traction by the PCB—electrode array cables was avoided by loosely attaching them to the surgical ring retractor. The mapping period was ˜15-20 min in each case, and two to three adjacent areas of stomach surface were mapped in each patient.

### Method of Analysis

Unipolar recordings were acquired from the vs via the ActiveTwo System (Biosemi, Amsterdam, The Netherlands), which was modified for passive recordings, at a recording frequency of 512 Hz. The common-mode sense (reference) electrode (CMS) was placed on the left shoulder, and the right-leg drive electrode (DRL) was placed on the right shoulder; slow wave recordings are referenced to the potential of the CMS electrode. The CMS and DRL were connected to standard 3M Ag/AgCl Red Dot cutaneous monitoring electrodes (3M, St Paul, Minn.). Each PCB was connected to the ActiveTwo via a 1.5 m 68-way ribbon cable, which was in turn fiber-optically connected to a notebook computer. The acquisition software was written in LabView 8.2 (National Instruments, Austin, Tex.).

Off-line signal analysis was performed in GEMS Software (The ‘Gastrointestinal Electrical Mapping Suite’; Auckland University, NZ). Signals were filtered by using a second-order Bessel low-pass filter with a cut-off threshold of 2 Hz. Individual slow wave events within the signal were detected using the falling edge variable threshold (FEVT) algorithm, which has been validated for this purpose. Slow waves were then partitioned into cycles using the REgion GROwing Using Polynomial Surface-estimate stabilization (REGROUPS) method. Isochronal activation maps were constructed according to our standard automated methods, in Matlab v.2006b (The Mathworks, Natick, Mass.).

### Results

### Normal Activity: Lower Corpus/Proximal Antrum:

In order to provide a baseline reference for the abnormal activities, an example of normal activity (activation, velocity and amplitude maps) is provided in FIG. 23 which shows:

FIG. 23a—mapping position; lower body/proximal antrum.
FIGS. 23b-d—activation time maps from 3 consecutive waves. Each black dot represents an electrode and each contrasting band (isochrone) shows the area of slow wave propagation per 2s of time. A stable activity pattern is shown that is typical of normal activity⁽¹¹⁾: propagation is aboral (in the direction indicated by the arrow, towards the pylorus). The isochronal bands are regular and align in the orthogonal organ axis (perpendicular to curvatures). The frequency is in the normal range (˜3.2 cpm).
FIGS. 23e-g—velocity maps from the same 3 cycles. Propagation direction is towards the pylorus. Velocity ranges from ˜2 mm/s to 4.5 mm/s, being faster at the greater curvature.
FIGS. 23h-j—amplitude maps from the same 3 cycles. Amplitudes are confined to a reasonably narrow range (˜200-600 μV).

### Patient A—Antral Dysrhythmia; Regular Tachygastria:

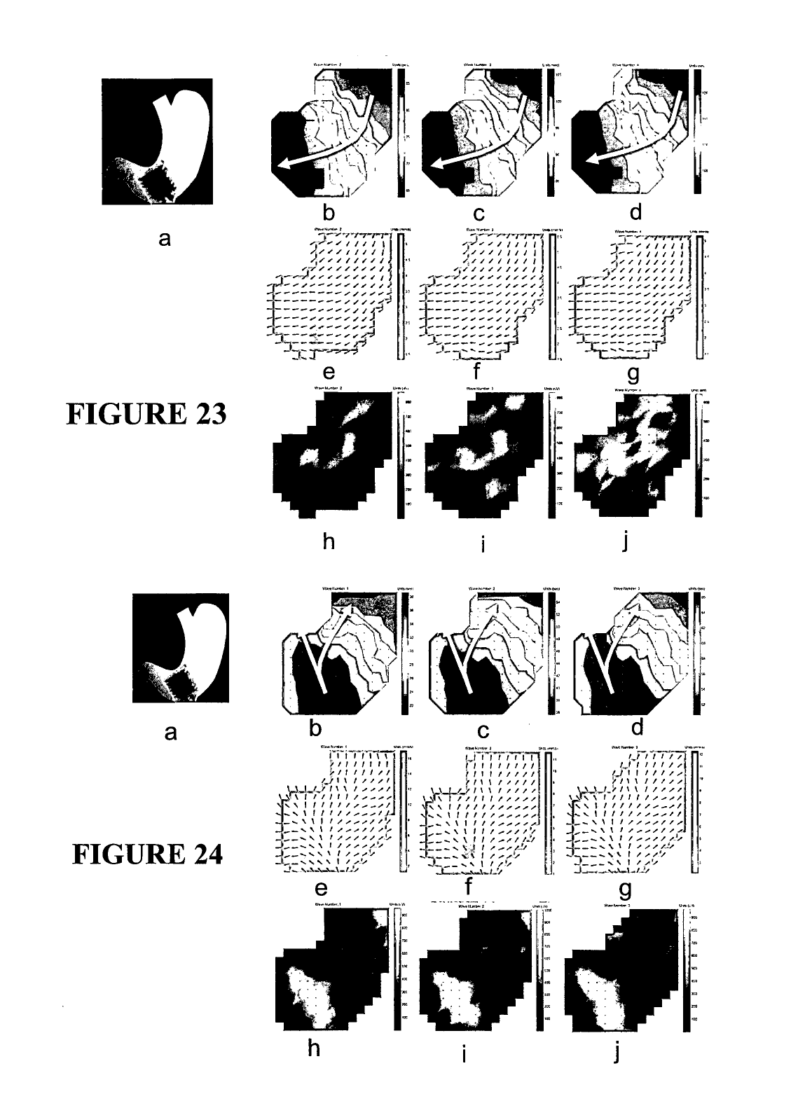

This patient provides an example of an antral tachygastria recorded in diabetic gastroparesis. FIG. 24 shows:

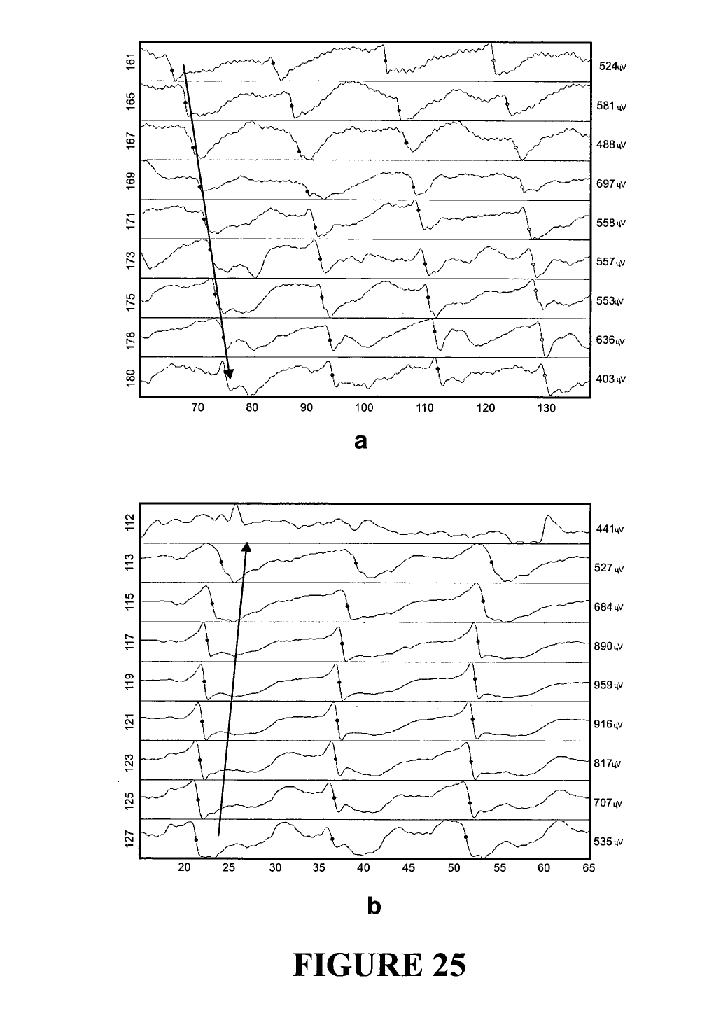

FIG. 24a—mapping position; lower body/proximal antrum (same as in normal control above).
FIGS. 24b-d—activation time maps are shown from 3 consecutive waves during tachygastria (frequency ˜4 cpm). A stable activity pattern is shown. Upward (retrograde) propagation is now present (in the direction indicated by the arrow, away from the pylorus), as well as circumferential propagation. The isochronal bands (2s intervals) are broad distally, indicating rapid propagation, toward the lesser curvature and spreading proximally.
FIGS. 24e-g—velocity maps from the same 3 cycles. Anisotropy is demonstrated, with the activity propagating in the circumferential direction being of higher velocity (˜10 mm/s) compared to the activity propagating longitudinally (˜3 mm/s).
FIGS. 24h-j—amplitude maps from the same 3 cycles. A high-amplitude region (800-900 uV) is associated with the area of high-velocity/circumferential propagation.
FIG. 25a show electrograms for Patient A prior to dysrhythmic onset and FIG. 25b shows electrograms for Patient A after onset of tachygastria (showing reversal of slow wave propagation).

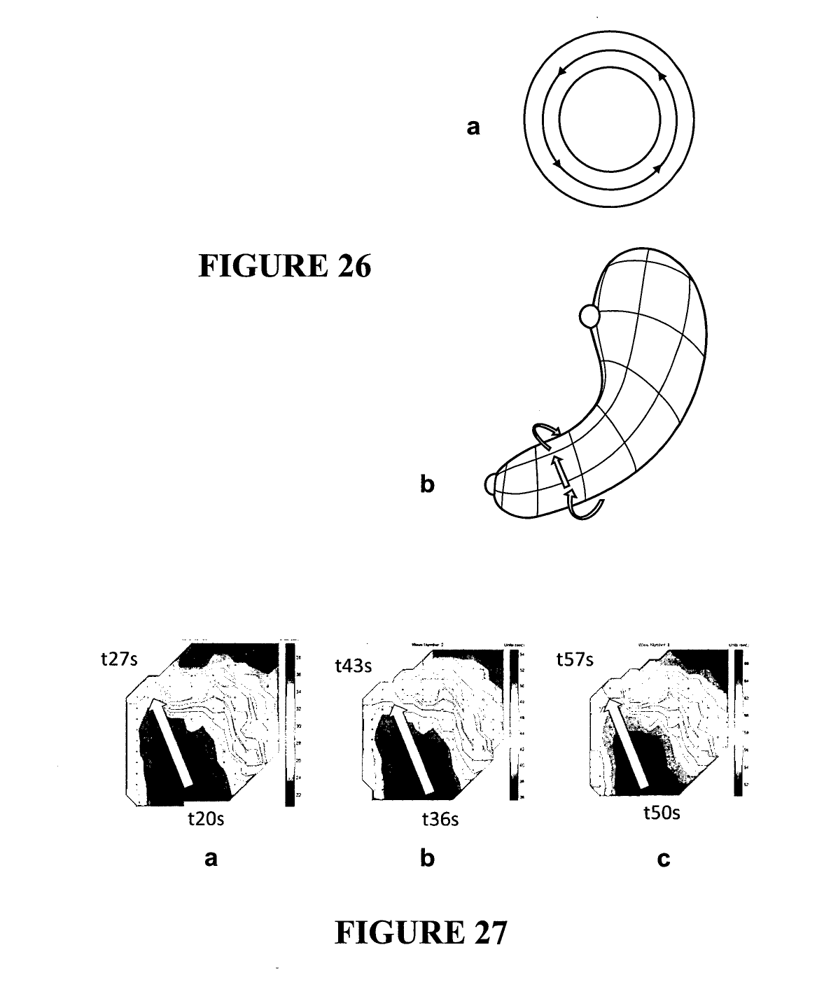

Observation of event times: Referring to FIGS. 26a and b and 27a-c the PCB—electrode array spans the antral width. Wave interval is 15 s. The wave takes ˜7 s to traverse the width of the array; if a similar amount of time is taken on the posterior stomach, and allowing 1 s for the unmapped curvature zones, then the frequency and timing fits with circumferential re-entry.

Two more examples of loop re-entry are next shown from the gastric corpus from two different gastroparesis patients than the antral tachygastria case. Activation and velocity maps are shown. Note that these events occurred at a frequency that would typically be considered to be ‘normal’, so the term ‘tachygastria’ should not be routinely applied to the loop re-entry mechanism.

### Patient B:

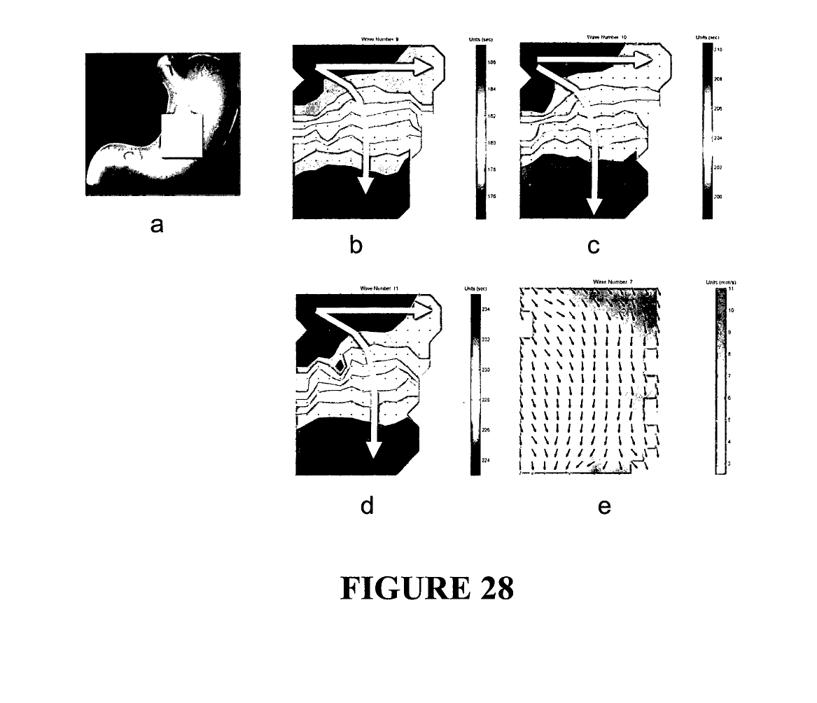

Referring to FIG. 28a, mapping was undertaken on the mid-body of the stomach (above left), and the activity was stable and consistent throughout. A consecutive series of activation maps and one velocity map are shown in FIGS. 28b-e, and these were typical of the recording. These maps show anisotropic propagation, with a rapid circumferential band of activity consistent with circumferential re-entry. This is abnormal for the human corpus.

### Patient C:

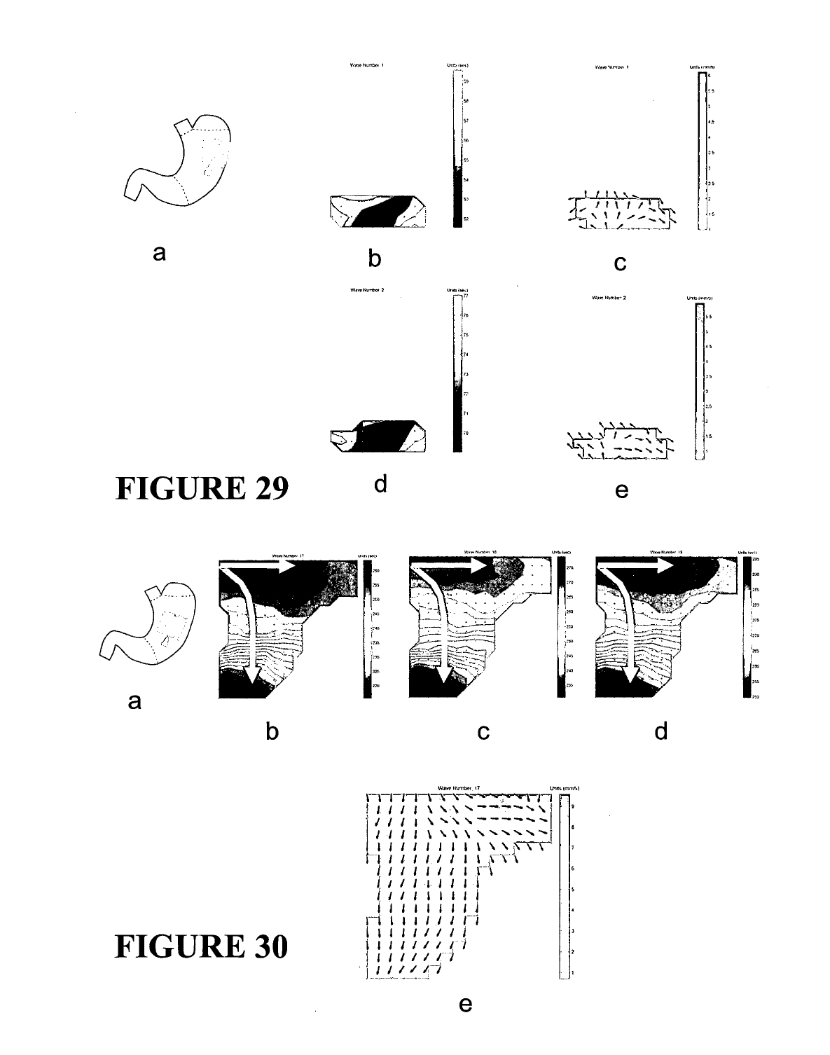

Mapping of the normal pacemaker region demonstrated only a small region of activation that failed to propagate >20 mm from the normal pacemaker site (ie, ‘exit block’)—see FIGS. 29a-e. Below the exit block, the following ectopic activity was observed in the corpus—see FIGS. 30a-e. These maps (similar to the Patient B) show anisotropic propagation with a rapid circumferential band of activity consistent with loop re-entry.

### Example 2

### Supporting Data from Small Intestine Mapping

Small intestine (SI) mapping studies show that that loop re-entry acts as a pacesetting mechanism in the GI tract, causing activity at higher than intrinsic frequencies and thereby inducing retrograde slow wave propagation.

### Method

In-vivo FIR serosal mapping was performed in five anesthetised weaner pigs using customized flexible PCB—electrode array) platforms (256 electrodes; 4 mm spacing, ˜35 cm²) that were wrapped around the circumferential curvature of the small intestine (SI). Silicone cradles were used to maintain PCB—electrode array contact over the curvature of the intestine. The electrode arrays were applied at representative intervals down the length of the intestine, from the proximal duodenum to the terminal ileum. Our analysis methods and GEMS Software (as described above) were utilized to characterise the spatiotemporal details of SW propagation and velocity in HR.

### Results

Circumferential re-entry loop activity was observed at multiple locations in multiple animals. In these instances, the electrode array was wrapped around the entire circumference of the intestine (except the mesenteric attachment). SW activity propagated orally and aborally from the circumferential re-entry loop sites and the frequency matched the expected formula:

$$f = \frac{{v}_{c}}{ϕ} ,$$

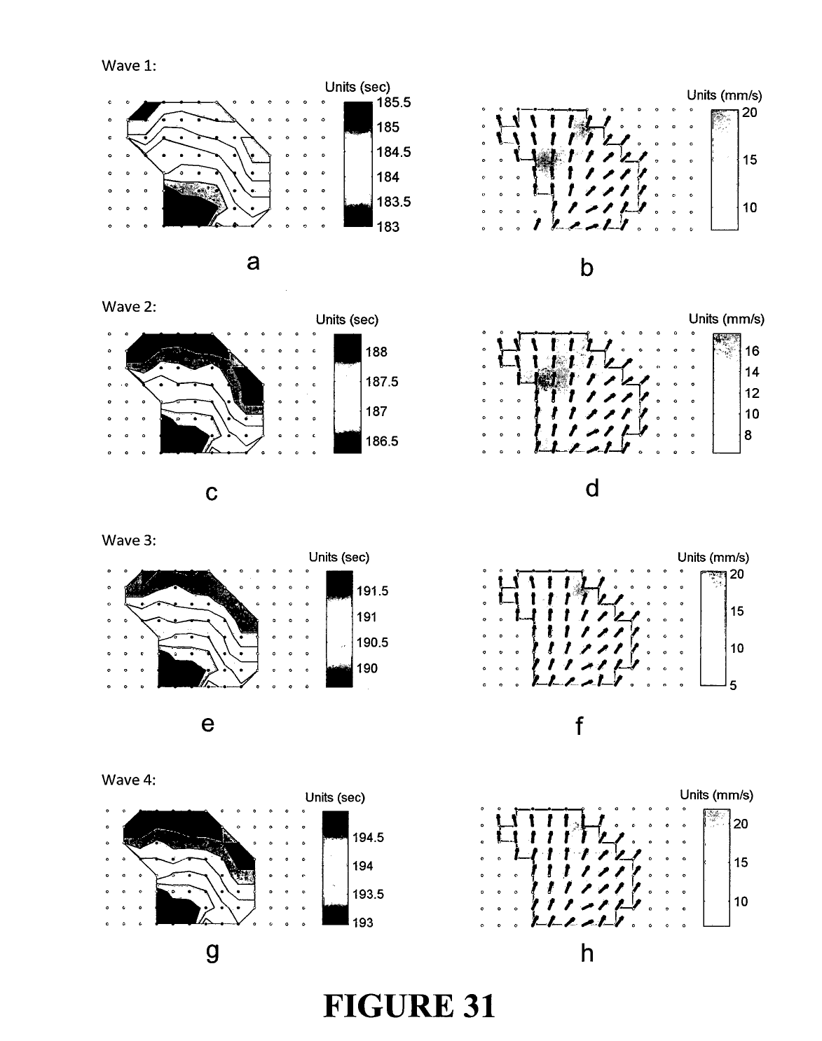

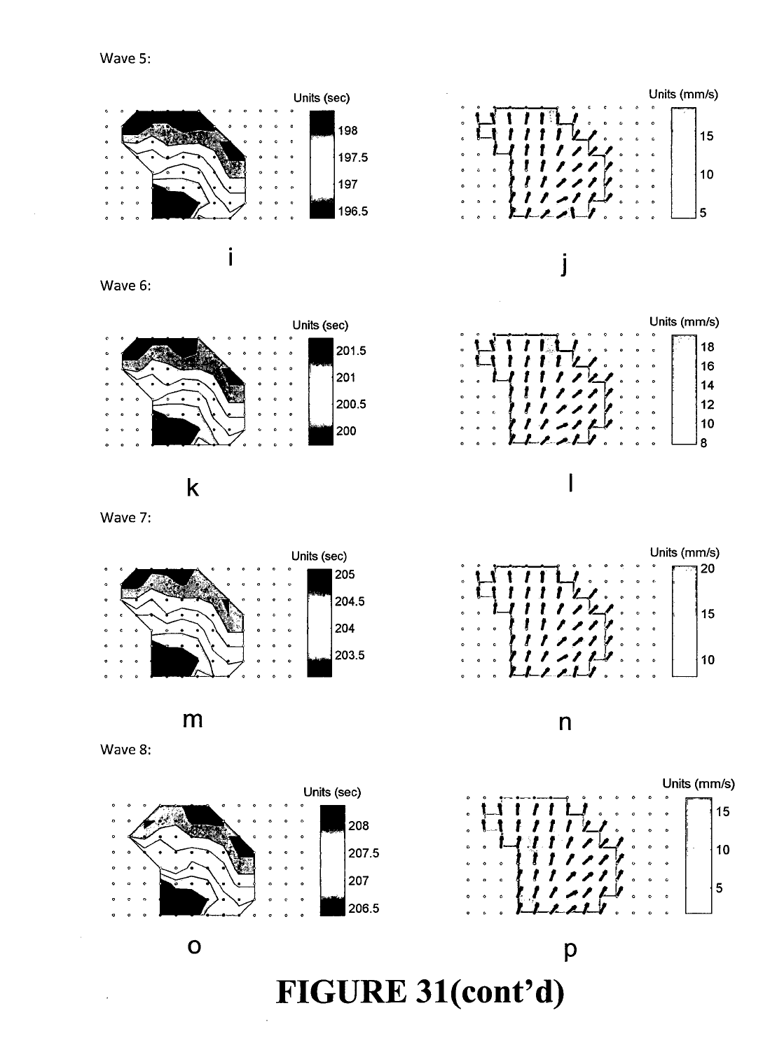

An example of stable SI circumferential re-entry loop activity is shown in FIG. 30, with activation and velocity maps. These are cropped to focus only on the loops. The orientation of the images is such that left is the oral direction and right is aboral. The top and bottom of the images is essentially the same location, i.e. the bottom wraps around the intestine to meet the top, with only a very small gap between. FIGS. 31a-p demonstrates stable re-entry loop activity with a frequency of about one loop every 3.5 seconds. The velocity maps show that the activity is propagating in the circumferential direction (bottom to top of the image represents looping around the circumference).

### Example 3

### Example of Abnormal Non-Re-entrant Circumferential Propagation Resulting from Incomplete Conduction Block

Apart from at the normal pacemaker site:
In normal activity, the average normalized gradient field of the activation times in the circumferential organ axis is nearly zero (say +/−˜0.05 for experimental error). Accordingly, the average normalized gradient field of the activation times in the organoaxial direction is normally greater than ˜0.95.
Abnormal activity in the above listed conditions is signalled by the presence of an increased normalized gradient field of the circumferential organ axis activity (typically to >0.1) likely along with an increase in the velocity specifically in that region where that increased gradient occurs likely along with a change in amplitude specifically in that, region where that increased gradient occurs.

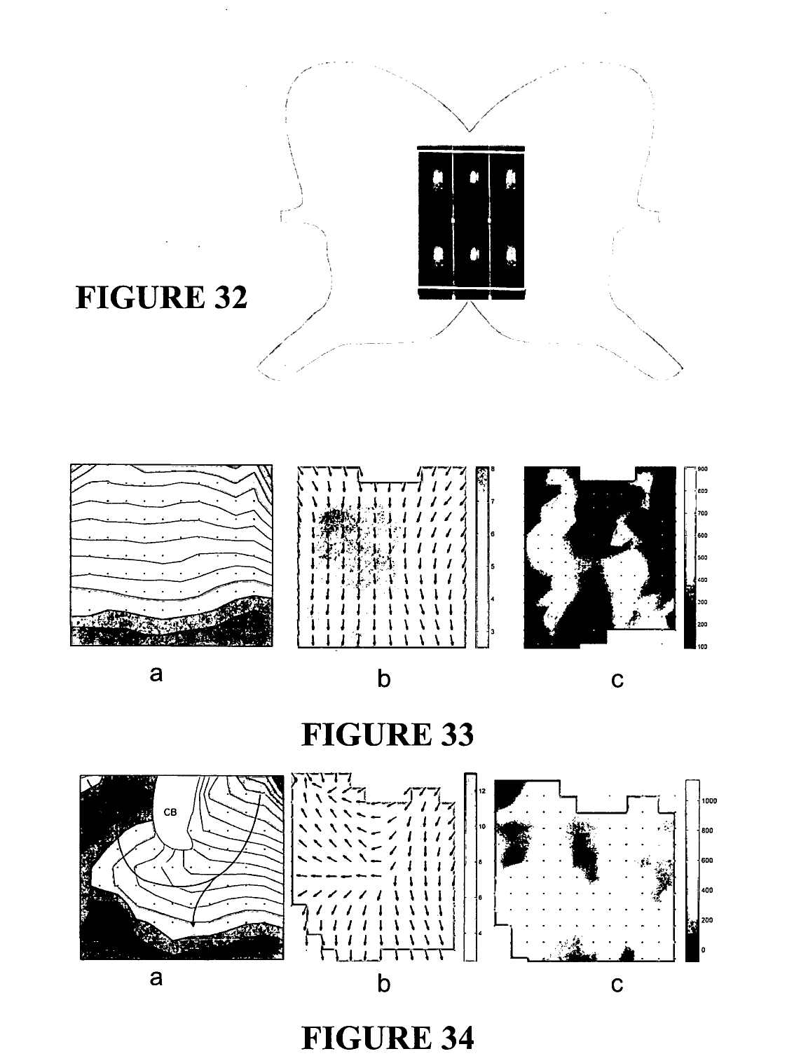

In a trial mapping methods generally as previously described were used on pigs. The recording position was over the greater curvature of the pig stomach, as shown in FIG. 32.

The normal control situation was as shown in FIG. 33, in which:

FIG. 33a is an isochrone map. Each contrasting band shows area of slow wave propagation per 1s of time. The activity propagates from the upper corpus (top of figure) to the lower corpus (bottom of figure). FIG. 33b is a velocity map (using described methods). Arrows show the propagation direction, the colours show the speed. The direction of propagation is uniform and toward the pylorus. The velocity scale is in mm/s and the mean velocity for the whole mapped field was 6.1+/−1.2 mm/s. The average normalized gradient field in the circumferential organ axis is near zero (0.01+/−0.02). The average normalized gradient field in the organoaxial direction is near 1 (0.97+/−0.05).
FIG. 33c is an amplitude map (scale is in uV). The mean amplitude for the mapped field was 357+/−191 uV.
Case of Incomplete Conduction Block (from Same Site)

A conduction block was induced by gastric handling. This is shown in FIG. 34 in which:

FIG. 34a is an isochrone map, again at 1 s increments. CB shows the region of the conduction block. The orientation of the isochrones has changed compared to the normal case.
FIG. 34b is a velocity map. The direction of propagation is no longer uniform, and includes circumferential propagation and upwards propagation around the block. The velocity scale is in mm/s and the total average velocity is now higher 6.5 mm/s+/−2 mm/s (p<0.01). In the circumferential direction, we can see that there is a patch of very high velocity, in the order of 2× as fast as normal longitudinal direction (ie ˜12 mm/s), associated with the circumferential propagation. The normalized gradient fields are now 0.33+/−0.42 in the circumferential organ axis and 0.57+/−0.61 in the organoaxial direction (p<0.001).

Referring to FIG. 34c the mean amplitude of the mapped field is now 456+/−236 uV. This is higher than in the control situation above by ˜27% (p<0.001). However, the amplitude increase is predominately in the area associated with the horizontal arrows on the velocity map (the circumferential propagation), which amplitudes are in the region of 800-1000 uv (2-3× the amplitude of normal activity).

Note, as per this example, that the abnormal amplitude and velocity ranges provided above are specific to the area of circumferential propagation, rather than the whole mapped field.

### Example 4

12 consecutive patients with medically-refractory diabetic (n=8) or idiopathic (n=4) gastroparesis, confirmed by standardized scintigraphy protocol testing (≧10% meal retention at 4 hours), underwent high-resolution serosal gastric mapping during gastric electrical stimulator implantation. Patients with malignancy, primary eating disorders, or pregnancy were excluded. The median age was 42 yrs (range: 30-62), median 4-hr gastric retention was 26% (range: 14-7.5%), median TSS (total symptom score on a 20 pt scale) was 16 (range: 13-20) and median BMI was 27 (range: 15.5-46).

All experiments were performed in the operating room following general anesthesia and upper midline laparotomy. The anesthetic methods used were similar to those used in another recent human study, in which 12 normal subjects underwent intra-operative mapping, and all showed exclusively normal slow wave activity.

### Methods of Mapping

HR mapping was performed using validated flexible printed circuit board (PCB) arrays. Each PCB had 0.3 mm electrode contacts, with 32 electrodes in a 16×2 configuration at 4 mm inter-electrode spacing, and in all cases eight PCBs were joined in parallel alignment with a sterile adhesive and used simultaneously (256 electrodes total; 16×16 array; 36 cm2). Mapping was undertaken immediately after laparotomy and prior to organ handling or stimulator placement. The PCBs were laid on the anterior stomach; the posterior surface was not mapped. The mapped positions were defined with reference to standard anatomical landmarks. Warm wet gauze was laid over the PCBs, the wound edges were approximated, and the cables were attached loosely to a retractor, ensuring they moved freely with respiratory excursion. The recording period was around 15 minutes in each case, with two or three adjacent gastric areas being mapped. Unipolar recordings were acquired at 256-512 Hz using a modified ActiveTwo System (Biosemi, The Netherlands). Reference electrodes were placed on the shoulders. Each PCB was connected to the ActiveTwo via a sterilized 1.5 m 68-way ribbon cable, and the ActiveTwo was fibre-optically connected to a computer. Acquisition software was written in Labview v8.2 (National Instruments, Tx).

Full-thickness gastric biopsies were taken from the anterior stomach and analysed for circular muscle interstitial cell of Cajal counts.

### Method of Analysis

All HR mapping analysis was performed in the Gastrointestinal Electrical Mapping Suite (GEMS) (v1.3). Recordings were down-sampled to 30 Hz, and filtered with a moving median filter for baseline correction, and a Savitzky-Golay filter for high-frequency noise. Slow wave activation times were identified using the FEVT algorithm, and clustered into discrete wavefronts (cycles) using the REGROUPS algorithm, with thorough manual review and correction of all automated results. Activation maps were generated using a further automated algorithm, and sites of conduction block (abnormal cessation of a propagating wavefront) were corrected using an additional automated step. Animations were prepared for the presented data segments. Frequency was determined by measuring and averaging the cycle intervals at all electrodes, and conduction velocities and extracellular amplitudes were calculated as follows. Velocity vector fields were generated using a finite difference approach, with interpolation and Gaussian filter smoothing functions, and visualized by overlaying arrows showing propagation direction on a ‘speed map’. Propagation directions were then decomposed into longitudinal and circumferential components. Amplitudes were calculated by identifying the zero-crossing of the first and second order signal derivatives of each event, before applying a peak-trough detection algorithm, and visualized by assigning a color gradient according to magnitude.

### Interpretation and Statistics

Normal HR reference data was previously established using similar methods in 12 patients with normal stomachs. This showed that slow waves propagate as successive ring wavefronts down the stomach, and circumferential propagation (wavefronts traveling transversely across the stomach) does not normally occur except at the pacemaker area. An example of normal activity is presented in FIGS. 34a-c. Prior to this work the dominant current view has been that slow wave propagation is regularly isotropic (proceeds in all directions at the same velocity). All recorded data was screened for deviations from normal activity by isochronal mapping and animation, and abnormalities were identified and quantified by frequency, rhythm (regular vs irregular), and spatial pattern. Tachygastria was defined as ≧3.7 c/min and bradygastria as ≦2.4 c/min. Tachygastric ‘bursts’ (lasting <1 minute) were distinguished from longer-lasting tachygastria (>1 min). Mean or median values are given for all outcomes, together with standard deviations (SD), standard errors (SE), or 95% confidence intervals, and Student's t-test was used for the statistical analyses (threshold p<0.05).

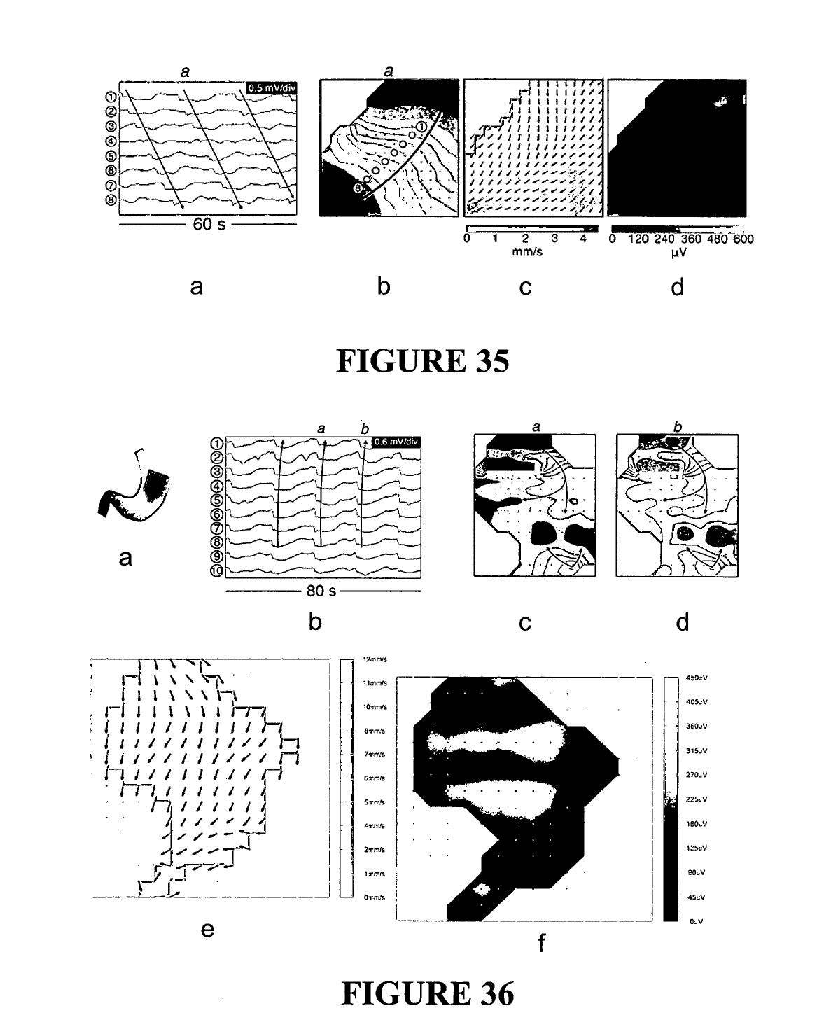

FIG. 35 shows electrograms from 8 channels (frequency normal; 3.2 (SD 0.1) c/min); FIG. 35b is an isochronal activation map of the wavefront (a) indicated in FIG. 35a, showing normal propagation—each dot represents an electrode, and each band shows the area of slow wave propagation per 2 s (the ‘isochronal interval’); FIG. 35c is a velocity field map of the same wavefront (a), showing the speed (spectrum) and direction (arrows) of the wavefront at each point on the array—p Propagation is faster nearer the greater curvature; and FIG. 35d is an amplitude map of the same wavefront (a).

### Results

Mean ICC counts were available and analyzed for 9/12 patients, and were substantially reduced in gastroparesis patients compared to the matched controls (2.3 (SE 0.3) vs 5.4 (SE 0.4) bodies/field; p<0.0001). The mean recording duration was 13.4 (SD 4.6) min/patient. Abnormal slow wave activity was recorded in 11/12 patients, and ranged from minor transient deviations from normal activity to persistent and highly disorganized patterns. The abnormalities were classified into either abnormalities of initiation (10/12 patients), or abnormalities of conduction (6/12), which often co-existed, and then subclassified by pattern, rhythm and rate according to the scheme illustrated in FIG. 21.

The emergence of rapid circumferential slow wave propagation was a consistent finding across: i) all cases of aberrant slow wave initiation, including both stable ectopic pacemakers and unstable ectopic focal activities, all cases of incomplete conduction block; and all cases of complete conduction block with escape. Across all patients with propagation direction data for comparison (n=8; corpus and proximal antrum inclusive), the velocity was faster during circumferential propagation than longitudinal propagation (7.3 (SE 0.9) vs 2.9 (SE 0.2) mm s−1; mean difference 4.4 mm s−1 [CI: 2.4, 6.4]; p=0.002). Extracellular amplitudes were also higher during circumferential propagation than longitudinal propagation (415 (SE 65) vs 170 (SE 25) μV; mean difference 245 μV [CI: 135, 360]; p=0.002).

### Example 5

### Example of a Conduction Block Causing Rapid High-Amplitude Circumferential Propagation

FIG. 36 illustrates a conduction block causing rapid high-amplitude circumferential propagation; FIG. 36a is a PCB—electrode array position diagram; FIG. 36b shows sample electrograms from the experimental recordings; FIGS. 36c and d are representative isochronal maps from a stable 5 minute recording period. Normal antegrade slow wave propagation is prevented by a conduction block (horizontal grey line). The block is incomplete and the wavefront is able to pass around it (top arrows). Beneath the block is a region of abnormally high velocity (˜7-10 mm/s) and amplitude (˜315-450 μV) activity, as demonstrated in the amplitude and velocity maps of FIGS. 36e and f (which follow the same event as shown in isochronal map d.

### Example 6

### Example of Ectopic Activities Showing Rapid Circumferential Propagation

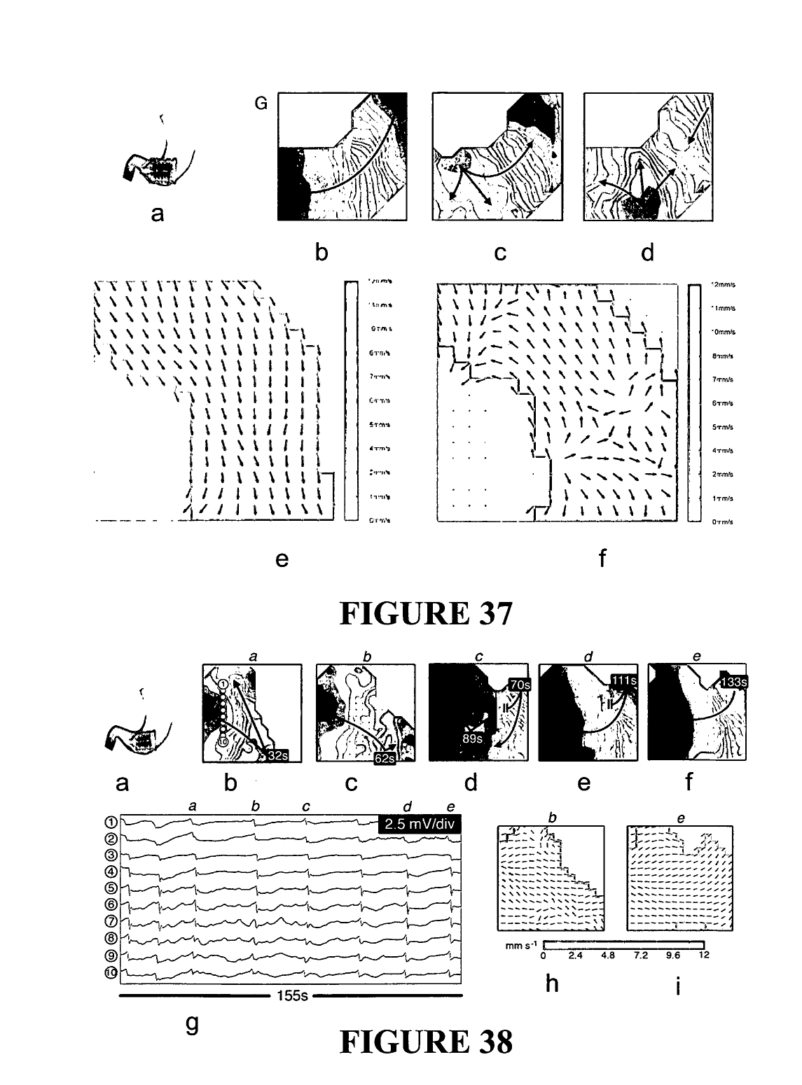

FIG. 37 illustrates ectopic activities showing rapid circumferential propagation (idiopathic gastroparesis). FIG. 37a is a PCB—electrode array position diagram. FIGS. 37b-d are isochronal maps showing: FIG. 37b—normal propagation; FIG. 37c—ectopic event arising near the lesser curvature of the corpus-antrum border; and FIG. 37d—ectopic event arising near the greater curvature. FIGS. 37e and f are velocity maps showing the emergence of rapid velocities in association with regions of circumferential conduction (FIG. 37f corresponds to event of FIG. 37d), compared to a normal velocity field during normal longitudinal propagation (FIG. 37e corresponds to the event of FIG. 37b) (mean 8.6 s.d. 3.4 mm/s vs 3.6 s.d 1.0 mm/s across several such waves; p<0.001).

### Example 7

### Example of Escape Events

FIG. 38 illustrates escape events (diabetic gastroparesis). FIG. 38a shows the PCB—electrode array position. FIGS. 38 b-f are isochronal maps showing: FIG. 38b—a conduction block (an area where normal propagation fails to pass) indicated by the vertical bar in the maps of FIG. 38d and FIG. 38e. In FIG. 38f, the normal wave successfully passes through this defective region, but at a very slow velocity. FIG. 38b and FIG. 38c show ‘escape events’ in which aberrant initiation of a new slow wave wavefront has occurred in an abnormal location, due to the a delay of the normal excitation. FIG. 38g shows example electrograms for the waves of FIGS. 38b-f from the mapping positions shown in FIG. 38b. FIGS. 38h & i are velocity maps corresponding to waves FIGS. 38b-f. In FIG. 38h there is an abnormally rapid area of slow wave propagation associated with circumferential propagation arising due to the escape event. The propagation velocity during normal longitudinal propagation in this region was mean 2.4 s.d 1.0 mm/s, compared to 5.3 s.d 2.7 mm/s during circumferential propagation associated with ectopic events; p<0.001). The extracellular amplitudes were also increased in association with the same circumferential propagation in escape events (mean 614 s.d. 370 μV vs 252 s.d. 159 μV; p<0.001).

### Example 8

### FEVT Activation Time Marking

Slow wave recordings of GI electrical activity were undertaken during surgery in pigs. Recordings were taken with both a high SNR 48 electrode array (resin-embedded, shielded, silver electrodes) and from a lower SNR electrode array (flexible PCBs; unshielded), from the anterior porcine gastric corpus. One 180 second representative data segment was selected from each of five animals: two segments from the high SNR array and three from the low SNR array. Unipolar recordings were acquired from the electrodes via the ActiveTwo System, at a recording frequency of 512 Hz. The common mode sense electrode was placed on the lower abdomen, and the right leg drive electrode on the hind leg. The electrodes array were connected to the ActiveTwo which was in turn connected to a notebook computer. The acquired signals were pre-processed by applying a second-order Butterworth digital band pass filter. The low frequency cutoff was set for 1 cpm (1/60 Hz); the high frequency cutoff was set to 60 cpm (1 Hz).

The slow wave ATs in each selected data segment were manually marked to provide a baseline for comparison. Within the electrode signal V(t), there are three dominant features of a slow wave event: (1) a small magnitude upstroke, immediately preceding (2) a fast, large magnitude, negative deflection (dV/dt˜=1 mV/s), followed by (3) a relatively long (5 s) plateau phase that decays slowly back to baseline. The fast negative-going transient corresponds with the depolarization wave front of the propagating slow wave, signalling the arrival of the slow wave at the recording electrode site. The point of most negative gradient during a slow wave was determined to be the AT.

Automated marking of the low SNR signals was carried out by the falling edge variable detection method. Some slow wave events exhibit a relatively fast recovery to baseline. This produces two large pulses in the transform detection signals, which can lead to erroneous double counting—the second mark in a set of two should not be marked. Such double-marking is precluded by imposing a criterion that distinct activation time events must be separated in time by a minimum value, termed the refractory period. Also, multiple slow wave events recorded by an electrode are not identical over time. For example, some pulses in a particular signal transform detection signals have larger amplitudes than the others. This amplitude difference can lead to missed detection of the smaller amplitude events. The FEVT algorithm implements a time-varying threshold (VT) to aid in the detection of ATs when recorded serosal waveforms may change over time.

Use was made of a falling-edge detector signal, E(t), to amplify the large-amplitude, high-frequency content associated only with negative deflections, suppressing positive-going transients in the process. It is formed by convolving the serosal electrical potential signal with an “edge-detector kernel” d_{Nedge}:E(t)=V(t)*d_{N}_{_{edge}} where * denotes the convolution operator. An edge-detector kernel (Sezan, Comput. Vis. Graph. Image Process. 49:36-51, 1990), was employed, which is formed from the convolution of a “smoother” with a “differencer”. N_{edge} defines the width of the kernel. A fixed value of N_{edge}=30, a 1-s wide kernel at fs =30 Hz, were chosen to correspond to the timescale of a typical large, negative transient. A falling edge (negative transient) in V(t) produces a positive deflection in E(t) (and vice-versa). When V(t) remains relatively constant, E(n) is approximately 0. Thus, E(t) is large and positive when V(t) contains a falling edge, and is negative for a rising edge. To help focus the slow wave detection algorithm on only the falling edges in V(t), the (element-wise) product of the smoothed detection signal S(t) was computed with the falling edge detection signal E(t), setting all negative values to zero. The resulting signal is termed the FEVT signal, F(t), which is thus summarized

$$F ( t ) = \{ \begin{matrix}S ( t ) E ( t ) & if S ( t ) E ( t ) \geq 0 \\ 0 & if S ( t ) E ( t ) < 0.\end{matrix}$$

To avoid slight variations in the waveforms leading to some events escaping detection, the FEVT method incorporated a time-varying detection threshold. Specifically, the time-varying threshold is based on the running median of the absolute deviation for time t using a window of half-width τ_{HW} centered at t for the FEVT signal, F(t):

$$\dot{σ} ( t ) = M \{ \vert F ( t - {τ}_{HW} ) - \hat{F ( t )} \vert … \vert F ( t + {τ}_{HW} ) - \hat{F ( t )} \} / 0.6745$$

where [symbol] is the sample mean of F(t) in the time range [t−τ_{HW}, t+τ_{HW] and M{•} denotes the sample median, as before. The variable threshold was then defined as: F}ₜₕᵣₑₛₕ=η×\hat{σ}(t), where η is a tunable parameter, as before. The moving median window was long enough to include the quiescent period in F(t) between the pulses of energy associated with the AT, but not so long that one slow wave can unduly influence the threshold defined for an event occurring much earlier or later. Values of 15, 30, and 45 s were used, which corresponds to about 1-2 full cycles 3 cpm gastric slow-wave waveform.

The FEVT method properly handled most problematic signals. For most electrodes, the FEVT detection algorithm succeeding in finding all ATs, without finding false positives. The overall performance of the FEVT algorithm was essentially invariant to the type of signal transform used when computing the FEVT signal. The FEVT detection signals contained large positive pulses corresponding to the negative-flanks of the corresponding electrode signal, while no such pulse was observed for positive-flank. The FEVT signals had a relatively high SNR. The time-varying threshold accommodates detection of ATs in an FEVT detection signal with a variable SNR. The FEVT algorithm was found suited to properly detect ATs in low SNR mucosally recorded signals.

### Example 9

### REGROUPS Cycle Clustering Method

Slow wave recordings were undertaken during surgery in pigs, and the recordings processed by the FEVT activation time marking method as described in Example 4. Recordings were taken with a low SNR array (flexible PCBs; unshielded), from the anterior porcine gastric corpus. Low SNR platforms were used because mucosal signals are typically of low SNR.

Four data sets (120 seconds duration) from four porcine subjects were selected because these segments represented a range of typical scenarios as follows:
Normal corpus propagation: Normally, gastric SWs propagate aborally as a transverse band (or ring) of activation, and consecutive wavefronts will be simultaneously detected by a large mapping array. A robust cycle partitioning algorithm must correctly determine which ATs belong to the distinct cycles, otherwise AT maps will be highly distorted and misleading. The first test case was from a corpus recordings on the greater curvature, featuring simultaneous, consecutive propagating wavefronts.
Normal pacemaker activity with peripheral region of quiescent tissue: Porcine SWs arise from a pacemaker area near the greater curvature of the mid-fundus; the upper and medial fundus are not activated. Robust analysis algorithms must correctly identify the concentric propagation, while demarcating the inactive regions. The second test case was recorded from the porcine gastric pacemaker site.
Abnormal propagation: Periodic abnormal SW behaviors are observed during porcine HR gastric mapping often characterized by retrograde propagation and/or ectopic pacemaking. Robust analysis methods must correctly identify abnormal propagation patterns. The third and fourth test cases were selected from data sets exhibiting retrograde propagation and ectopic pacemaking, recorded from the upper corpus/distal fundus. Importantly, the latter three of these test cases also had patchy data quality, which results from suboptimal or obstructed electrode contact, or due to interfering signals (e.g., respiration artifacts).
Competing pacemakers/clashing wavefronts: When more than one region acts as a pacemaker, the multiple corresponding wavefronts generated by them will collide. Such dysrhtymic activity may correspond to clinically diagnosable conditions. Robust analysis methods must correctly identify that a single cycle contains multiple clashing wavefronts.

The REGROUPS algorithm works by clustering (x, y, t) points representing ATs into groups that represent independent cycles ((x, y) denotes the position of an electrode site (relative to an arbitrary reference), and t denotes an AT marked at that site). The algorithm is initialized by creating a master list of all marked ATs, and selecting the master seed electrode site in automated fashion (see below). A queue containing the (x; y) positions of nearby sites is established. A “nearby” site was defined as falling within a distance \sqrt{2}dₘᵢₙ of the seed electrode, where dₘᵢₙ denotes the minimum distance between the seed site and the closest site containing (at least) one AT. The factor of \sqrt{2} essentially defines a circular search radius (for a square lattice array) to include sites located diagonal to the seed. dₘᵢₙ is not necessarily equal to the inter-electrode spacing (although it often will be), enabling the algorithm to successfully “jump” across local patches of missing data.

REGROUPS also employs an iterative “flood fill” or “region growing” procedure. The first queue entry (electrode site) becomes the current seed, and all ATs at that site, AT(x; y; j) (where j=1, . . . , J indexes the marked ATs), are tested for membership. A point (x; y; t) in AT(x; y; j) is assigned membership to the cluster (or not) based on comparison to an estimated AT, Tₑₛₜ. If the difference is small enough, the AT which minimizes the estimate error is assigned membership to the cluster:

$${min}_{j} \vert AT ( x , y , j ) - {T}_{est} \leq Δ {t}_{max} .$$

Once assigned, membership is never revoked. A point can be assigned membership to only one cluster (at most): Upon assignment, that (x; y; t) point is removed from master list of ATs so that is never tested again during the remainder of the clustering process. If the tested point is clustered, all of its nearby neighbors are added to the back of the queue, if they are not already in it. If the tested point is not clustered, it may be tested again for membership only after new cluster has initialized (a new activation time surface is calculated) at the next iteration. This restriction forces all wavefronts to be independent. Regardless of whether any point was added to the cluster, the current seed is removed from the queue, and the next queue element becomes the current seed. Thus, the region in (x, y, t) space representing an independent cycle grows, terminating when the queue of nearby points becomes empty. At this stage, the cluster contains all ATs from one cycle. The same process is repeated anew to identify another independent cycle, starting with the next sequential AT marked at the master seed. Each iteration produces a cluster of (x, y, t) points, which represent the dynamics of an independent cycle. Points which are not assigned membership to any cluster are termed “orphans.”

A step is to implement a 2nd-order polynomial surface, T(x, y), to act as a continuously updating spatiotemporal filter, where: T(x,y)=p₁x²+p₂y²+p₃xy+p₄x+p₅y+p_{G}.

Using only the (x, y, t) already in cluster, the vector of coefficients that defines the surface, p=[p1, p2, p3, p4, p5, p6], is computed using a previously described least-squares-fitting procedure: P=(A^{T}A)⁻¹At where A is a matrix whose rows are created using the (x, y) electrode positions of points already in the cluster: [x², y², xy, x, y, 1]; and t is a column vector containing the corresponding ATs marked at those electrode sites. Having solved for the vector of coefficients p that defines the polynomial surface, an estimate of the AT at a nearby site (xₙ, yₙ) can be obtained by simply extending the surface into that region: Tₑₛₜ=T(xₙ, yₙ). The coefficients describing the surface, p, are automatically updated every time another point is added to the cluster. Therefore, the data set at hand determines the form of the polynomial surface, making it substantially more robust and more widely-applicable for distinguishing independent cycles in a variety of SW behaviors. At least 6 points are required to obtain a fully determined system of equations, so prior to switching on the polynomial surface estimation, Tₑₛₜ is computed as the mean of the ATs of the points already assigned membership in the cluster. In practice, we found the algorithm performs best when the polynomial surface estimation is switched on when the cluster size reaches a “critical mass” of at N_{or it}≧12 points, which is on the order of frac110 the total number of electrode sites on the recording platform (data not shown). If the critical mass is too small, then the surface was overfit to a small core of points, yielding a poor description of the propagation pattern across the entire electrode array. On the other hand, if the critical mass was too large, then the technique fails to utilize information about the velocity gradient at the wavefront boundary, which is critical for the success of the algorithm (other spatiotemporal filters may be introduced into the software to aid detection of different electrical patterns).

The outcome of clustering is dependent on the initial seed selection, particularly when the data quality is patchy (sparse). Seed selection was automated such that the seed was chosen to be at an electrode position (x, y)_{seed} which is typically embedded in a region providing the maximal density of information about the propagating wavefront:
For each electrode site, tally N(x, y), the total number of ATs detected at an electrode site location (x, y).
Compute the center of mass (CM) (x_{CM}, y_{CM}) using the entries of N(x, y):

$${x}_{CM} = \frac{{\sum }_{i} N ( {x}_{i} , {y}_{i} ) {x}_{i}}{{\sum }_{i} N ( {x}_{i} , {y}_{i} )}$$

where the sum is taken over all electrode sites, indexed by i. The y-coordinate y_{CM} is similarly computed.
Check if (x_{CM}, y_{CM}) corresponds to the coordinates of an electrode with marked ATs. If yes, then the seed is selected to be the CM. If not, move the seed to the closest electrode site meeting this condition. In practice, the seed is usually selected to be at the CM.

Isochronal slow wave activation maps were generated. Control and experimental arms were developed to compare completely automated versus completely manual results, starting from raw data and ending with AT maps. This approach therefore sought to validate the FEVT-REGROUPS-Automated-Isochronal-Mapping pipeline, to demonstrate real world practicability of the complete system:
experimental arm: ATs were identified via the FEVT method. The REGROUPS and automated isochronal mapping algorithms were applied to each FEVT auto-marked data set to identify the first 5 consecutive SW cycles.
control arm: ATs were manually assessed and marked by a fully blinded manual marker. ATs were manually marked at the apparent point of steepest negative slope. The resulting ATs were then manually partitioned to identify the first 5 consecutive SW cycles, and resultant isochronal maps generated. The manually generated maps were considered to be the standard for comparison.

Quantitative comparison: The automated results were quantitatively compared to the manually-derived results in terms of AT mapping a) area of coverage, and b) isochronal timing accuracy. The REGROUPS results showed strong similarity to the manual results with comparable isochronal intervals and orientations, comparable map coverage, and a high consistency between cycles. For normal pacemaker activity and peripheral quiescent region the REGROUPS results proved similar to the manual marking results with comparable isochronal intervals, orientations, and consistency between cycles, and similar spatial map coverage. For abnormal activity the manual maps and REGROUPS maps were highly comparable in terms of isochronal intervals and orientations. The REGROUPS consistently demonstrated slightly greater spatial coverage than the manual maps, extending proximally with a physiologically-consistent activation pattern.

The foregoing describes the invention including embodiments and examples thereof, and alterations and modifications are intended to be incorporated in the scope hereof as defined in the accompanying claims.

1. A gastrointestinal-electrical activity mapping and/or signal analysis system arranged to receive and process electrical signals from one or more electrodes and spatially map GI smooth muscle electrical activity at said section of the GI tract and/or identify as indicative of disease including (but not limited to) gastroparesis and/or functional dyspepsia or as useful in the diagnosis of disease mechanisms in gastro-oesophageal reflux disease, or nausea and vomiting disorders, and other gastro-intestinal motility disorders any one of or any of in combination:
propagation of re-entrant or non re-entrant GI electrical activity upwardly within the GI tract, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

2. A system according to claim 1 arranged to identify propagation of re-entrant or non re-entrant GI electrical activity upwardly within the GI tract from the pylorus or antrum or other lower part of the corpus of the stomach in a direction towards an upper part of the corpus at frequency above about 3.7 cycles per minute (cpm) or 4 cpm, as a result of a re-entrant propagation around the GI tract.

3. A system according to claim 1 arranged to identify propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum.

4. A system according to claim 1 arranged to identify propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,

5. A system according to claim 1 arranged to identify propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,

6. A system according to claim 1 arranged to identify propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, as a result of a conduction problem including a partial or complete conduction block, and other than at the normal pacemaker site in the stomach.

7. A system according to claim 1 arranged to identify propagation of non re-entrant GI electrical activity circumferentially within the GI tract, at a velocity above 2 times or between 1.5 and 3.5 times normal velocity and/or with an amplitude of above 2 times or between 1.5 and 3.5 times normal amplitude, as a result of a focal event or events or ectopic activity or activities or as a result of an escape activity, and other than at the normal pacemaker site in the stomach.

8. A system according to claim 1 wherein the signal analysis and mapping system is arranged to spatially map and visually display to a user GI electrical activity in real time or near-real time.

9. A system according to claim 1 wherein the signal analysis and mapping system is arranged to map GI electrical activity as an activation time map of the GI electrical activity.

10. A system according to claim 1 wherein the signal analysis and mapping system is arranged to map GI electrical activity as a velocity map indicative of the direction and speed of the GI electrical activity.

11. A system according to claim 1 wherein the signal analysis and mapping system is arranged to map GI electrical activity as an amplitude map of the amplitude of the GI electrical activity.

12. A system according to claim 1 wherein the signal analysis and mapping system is arranged to map the GI electrical activity as a contour plot of the GI electrical activity.

13. A system according to claim 1 wherein the signal analysis and mapping system is arranged to map the GI electrical activity on an anatomical model of at least the section of the GI tract.

14. A system according to claim 1 wherein the signal analysis and mapping system comprises a database of one or more anatomical model geometries of one or more sections of the GI tract.

15. A system according to claim 1 wherein the signal analysis and processing system is arranged to analyse the GI electrical activity for events indicative of GI slow waves and then to cluster the detected events into groups each relating to a common GI slow wave based on temporal closeness.

16. A system according to claim 15 wherein the signal analysis and processing system is arranged to analyse the GI electrical activity for events indicative of slow waves by falling edge detection and a time varying threshold.

17. A system according to claim 15 wherein the signal processing and mapping system is arranged to cluster the detected events by a region growing using polynomial surface estimate stabilization method.

18. A system according to claim 1 arranged to identify and report abnormal GI electrical activity.

19. A system according to claim 1 arranged to display the position of electrodes of the array on a map or anatomical model of at least a section of the GI tract.

20. A system according to claim 1 arranged to map the GI electrical activity as an animation.

21. A system according to claim 1 comprising a catheter insertable into the gastro-intestinal (GI) tract, or other electrode-carrier for contacting an exterior serosal surface of the GI tract, and comprising an array of electrodes for contacting a surface of a section of the GI tract to detect electrical potentials at multiple electrodes.

22. A system according to claim 21 wherein the catheter comprises an electrode carrier carrying on an exterior surface the array of electrodes and expandable when in place to cause the electrodes to contact the interior surface of the GI tract.

23. A method for analysing and/or mapping GI electrical activity which comprises acquiring electrical potentials from at least one electrode contacting a surface of a section of the GI tract and spatially mapping from the electrical signals GI electrical activity at said section of the GI tract, and identifying as indicative of disease including (but not limited to) gastroparesis and/or functional dyspepsia or as useful in the diagnosis of disease mechanisms in gastro-oesophageal reflux disease, or nausea and vomiting disorders and other gastro-intestinal motility disorders any one of or any of in combination:
propagation of re-entrant or non re-entrant GI electrical activity upwardly within the GI tract, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach.

24-39. (canceled)

40. A gastrointestinal-electrical activity mapping and/or signal analysis system arranged to receive and process electrical signals from one or more electrodes and spatially map GI smooth muscle electrical activity at said section of the GI tract and/or identify as indicative of disease including (but not limited to) gastroparesis and/or functional dyspepsia or as useful in the diagnosis of disease mechanisms in gastro-oesophageal reflux disease, or nausea and vomiting disorders, and other gastro-intestinal motility disorders any one of or any of in combination:
propagation of re-entrant or non re-entrant GI electrical activity upwardly within the GI tract, optionally at frequency above about 3.7 cycles per minute (cpm) or 4 cpm, as a result of a re-entrant propagation around the GI tract,
propagation of re-entrant or non re-entrant GI electrical activity circumferentially within the GI tract other than at the normal pacemaker site in the stomach,
propagation of re-entrant GI electrical activity substantially only circumferentially within the GI tract,
propagation of non re-entrant GI electrical activity circumferentially within the GI tract at a velocity and/or amplitude higher than the normal (non re-entrant) GI electrical activity in the GI tract and/or at a higher circumferential velocity than longitudinal velocity in the GI tract, other than at the normal pacemaker site in the stomach,
propagation of re-entrant GI electrical activity substantially only circumferentially within the GI tract and/or at a higher circumferential velocity than longitudinal velocity in the GI tract, other than at the normal pacemaker site in the stomach,
propagation of re-entrant or non re-entrant GI electrical activity circumferentially in the corpus of the stomach at a velocity above 2 times or between 1.5 and 3.5 times normal velocity in the corpus and/or between 1.25 and 2.5 times normal velocity in the antrum, other than at the normal pacemaker site in the stomach, and/or with an amplitude of above 2 times or between 1.5 and 3.5 times the normal amplitude in the corpus,
propagation of re-entrant or non re-entrant GI electrical activity circumferentially in the antrum of the stomach at a velocity above 1.5 times or between 1.5 and 3.5 times normal velocity in the antrum, and with an amplitude of above 2 times or between 1.5 and 3.5 times the normal amplitude in the antrum,
propagation of non re-entrant GI electrical activity substantially only circumferentially within the GI tract at a velocity above 2 times or between 1.5 and 3.5 times normal velocity and/or with an amplitude of above 2 times or between 1.5 and 3.5 times normal amplitude, and other than at the normal pacemaker site in the stomach,
propagation of non re-entran) GI electrical activity circumferentially within the GI tract, at a velocity above 2 times or between 1.5 and 3.5 times normal velocity and/or with an amplitude of above 2 times or between 1.5 and 3.5 times normal amplitude, and other than at the normal pacemaker site in the stomach,
propagation of any of the above defined GI electrical activity arising from one GI slow wave passing more than once through a same path of tissue conduction, and
propagation of any of the above defined GI electrical activity at an amplitude and/or velocity higher than the normal (non-reentrant) GI electrical activity, other than at the normal pacemaker site in the stomach,
the signal analysis and mapping system being arranged to spatially map and visually display to a user GI electrical activity in real time or near-real time at least one of:
an activation time map of the GI electrical activity,
a velocity map indicative of the direction and speed of the GI electrical activity,
an amplitude map of the amplitude of the GI electrical activity, and
a contour plot of the GI electrical activity,
as an animation on an anatomical model of at least the section of the GI tract.
